(19) United States
(12) Patent Application Publication
(10) Pub. No.: US 2024/0024667 A1
(43) Pub. Date: Jan. 25, 2024

(54) SYSTEM AND METHOD FOR NERVE STIMULATION

(71) Applicant: RBI Medical, Worcester, MA (US)

(72) Inventors: Jayme Coates, Worcester, MA (US); David Constantine, Sudbury, MA (US); Mario Romero-Ortega, Coppell, TX (US); Mark Tauer, Worcester, MA (US)

(21) Appl. No.: 18/225,129

(22) Filed: Jul. 22, 2023

Related U.S. Application Data
(60) Provisional application No. 63/391,574, filed on Jul. 22, 2022.
(60) Provisional application No. 63/391,584, filed on Jul. 22, 2022.

(51) Int. Cl. A61N 1/05; A61N 1/36

(52) U.S. Cl. CPC A61N 1/0558; A61N 1/0553; A61N 1/36007

(57) ABSTRACT

A neuromodulation device includes a movable arm coupled with a main body. The movable arm is configured to transition between an open configuration and a closed configuration. The movable arm is biased towards the closed configuration. A nerve stimulation chamber is defined at least in part by the movable arm. The nerve stimulation chamber is configured to retain a nerve therein. The device has an open channel through which the nerve travels. The channel is defined at least in part by the movable arm. The size and shape of the chamber and the channel are adjustable by the movement of the arm in response to contact with the nerve. The channel may be continuously axial along a longitudinal axis of the channel when the movable arm is in the closed configuration. The chamber has an electrode.

### PRIORITY

This patent application claims priority from provisional U.S. patent application Nos. 63/391,574, and 63/391,584, both filed Jul. 22, 2022, the disclosures both of which are incorporated herein, in their entirety, by reference.

### FIELD OF THE INVENTION

Illustrative embodiments of the invention generally relate to pelvic organ function and, more particularly, various embodiments of the invention relate to selective neuromodulation for controlling bladder and pelvic floor function.

### BACKGROUND OF THE INVENTION

Suffered by millions of people in the US and worldwide, overactive bladder (“OAB”) can cause debilitating urgent and frequent urination with or without urinary incontinence. Puzzling to many in the art, OAB is associated with nocturia, but in the absence of urinary tract infection or other obvious pathological conditions. The pathophysiology and etiology of idiopathic OAB remains unknown, but it seems to be related to bladder detrusor muscle dysfunction, or detrusor overactivity. The current pharmacological treatment includes oral anti-muscarinics or oral β3-adrenoceptor agonists. Such treatments have significant drawbacks as they have limited efficacy and tolerability, and cause significant side effects including dry mouth, dry eyes, constipation, tachycardia, and potential long-term cognitive effects. Consequently, studies have shown that the majority of OAB patients stop taking these drugs within 6-12 months of treatment.

Another urinary problem, known as “stress urinary incontinence” (“SUI”), occurs when urine leaks out with sudden pressure on the bladder and urethra, causing the sphincter muscles to open briefly. With mild SUI, pressure may be from sudden forceful activities, such as moderate exercise, sneezing, laughing or coughing. If SUI is more severe, however, urine may leak with less forceful activities, such as standing up, walking or bending over. Urinary “accidents” like this can range from a few drops of urine to enough to soak through your clothes.

Those in the art typically treat both OAB and SUI with different treatment regimens.

### SUMMARY OF VARIOUS EMBODIMENTS

In accordance with an embodiment, a neuromodulation device includes a main body having a hermetically sealed housing containing electronics therein. The main body has a buffer layer at least partially encapsulating the hermetically sealed housing. A movable arm is coupled with the main body. The movable arm is configured to transition between an open configuration and a closed configuration. The movable arm is biased towards the closed configuration. A nerve stimulation chamber is defined at least in part by the movable arm. The nerve stimulation chamber is configured to retain a nerve therein. The size and shape of the chamber is adjustable by the movement of the arm in response to contact with the nerve. The device has an open channel through which the nerve travels. The channel is defined at least in part by the movable arm. The size and shape of the channel are adjustable by the movement of the arm in response to contact with the nerve. The channel may be continuously axial along a longitudinal axis of the channel when the movable arm is in the closed configuration. The chamber has an electrode.

Some embodiments include a second arm. The second arm may be configured to transition between an open configuration and a closed configuration. The second arm may be biased towards the closed configuration. Movement of the second movable arm may adjust a size of the chamber and/or a size of the channel. The movable arm may be configured to atraumatically retain a nerve having a resting maximum cross-sectional dimension of between 0.5 mm and 4 mm is in the chamber and when the movable arm is biased towards the closed configuration.

Among other things, the chamber may include one or more nerve relief spaces defined by the movable arm. The movable arm may also include one or more bending points. A distal end of the electrode may be embedded within the movable arm. A continuous feedthrough electric conductor wire may extend through the hermetically sealed housing to form the electrode.

In various embodiments, the channel is non-linear. The chamber may be configured so that the electrode is curved and sized to contact at least 20% of the perimeter of a nerve having a maximum cross-sectional dimension of between 0.5 mm and 4 mm. The channel may be both non-linear and continuously axial.

In accordance with another embodiments, a method of stimulating a nerve provides a neuromodulation device. The device has a main body including a hermetically sealed housing containing electronics therein, and a buffer layer at least partially encapsulating the hermetically sealed housing. A movable arm is coupled with the main body. The movable arm is configured to transition between an open configuration and a closed configuration. The movable arm is biased towards the closed configuration. A nerve stimulation chamber is defined at least in part by the movable arm. The nerve stimulation chamber is configured to retain a nerve therein. The size and shape of the chamber is adjustable by the movement of the arm. A channel through which the nerve travels is defined at least in part by the movable arm. The size and shape of the channel is adjustable by the movement of the arm. The channel is continuously axial along a longitudinal axis of the channel. The device may include an electrode within the chamber, The method reduces a maximum cross-sectional dimension of a nerve to define a stretched nerve having a reduced cross-sectional dimension. The stretched nerve is moved through the channel along the central axis of the channel. The nerve is positioned in the chamber and the cross-sectional dimension of the stretched nerve is increased.

In various embodiments, the nerve is stimulated using the electrode. A variety of control signals are used to control the stimulation parameters.

In accordance with another embodiment, a method stimulates a nerve. The method provides a device having a main body including a hermetically sealed housing containing electronics therein. The device has an arm formed of an elastomeric material. A channel through which the nerve travels is defined at least in part by the arm. The channel has a continuously axial longitudinal axis at rest. A nerve stimulation chamber is defined at least in part by the arm. The nerve stimulation chamber is configured to retain a nerve therein. The device has an electrode in the chamber. The method reduces a maximum cross-sectional dimension of a nerve to define a stretched nerve having a reduced cross-sectional dimension. The reduction in maximum cross-sectional dimension being less than 50% of the diameter of the undeformed nerve. The stretched nerve is moved through the channel along the central axis, such that the channel walls apply less than 6.7 kPa of pressure to the nerve at any given point. The nerve is positioned in the chamber, and the cross-sectional dimension of the stretched nerve is increased. At least 20% of the perimeter of the nerve is maintained in contact with the electrode.

In various embodiments, the nerve may have a different cross-sectional shape when in the chamber, but the cross-sectional area may be substantially the same as the undeformed nerve.

Some embodiments may reduce a cross-sectional dimension of the nerve in the chamber to define a stretched nerve having a second reduced cross-sectional dimension. The reduction in maximum cross-sectional dimension may be less than 50% of the maximum cross-sectional dimension. The stretched nerve may be moved through the channel along the central axis to remove the nerve from the device, such that the channel walls apply less than 6.7 kPa of pressure to the nerve.

Various embodiments may maintain at least 20% of the perimeter of the nerve in contact with the electrode while provides less than 4 kPa of retention pressure to the nerve. The nerve may have an undeformed diameter of between 0.5 mm and 4 mm. The cross-sectional area of the nerve positioned in the chamber may be greater than 50% of the cross-sectional area of the undeformed nerve. In some embodiments, the cross-sectional area of the nerve in the chamber may be equivalent to the cross-sectional area of the undeformed nerve. To that end, the chamber may include nerve relief spaces.

The arm may be movable between an open configuration and a closed configuration. The arm may be biased towards the closed configuration, such that the size of the chamber and/or the channel is adjustable by movement of the arm. The arm may have one or more bending portions, such that the size of the chamber is adjustable by bending of the arm. The arm may include a buffer material that is configured to deform with less than 6.7 kPa of pressure, such that the size of the chamber and/or the channel is adjustable by deformation of the arm. One or more arms may form a continuously axial channel through which the nerve travels. The channel may be defined at least in part by the arm. The channel may be non-linear along its central axis.

In various embodiments, the buffer layer may be formed from silicone. The package may be formed from glass, ceramic, alumina, zirconium, and/or plastic. A continuous feedthrough conductor may extend through the package, the buffer lay, and through the movable arm. The feedthrough conductor may form the electrode. The electrode may thus advantageously be unwelded/unjointed.

In accordance with another embodiment, a movable arm is formed by an elastomeric material. The movable arm is configured to transition between an open configuration and a closed configuration. The movable arm being biased towards the closed configuration. The movable arm at least in part defines a chamber and a channel. The channel having a gap of less than 1 mm. The movable arm is configured to allow passage of nerves through the channel for nerves having diameters of between 1 mm and 3 mm while providing less than 6.7 kPa of pressure to the nerve. The movable arm is further configured to provide less than 4 kPa of pressure to the nerve as it is stimulated by an electrode in the chamber.

The movable arm may be configured to press against the nerve in the chamber to deform the nerve, but not to reduce a cross-sectional area of the nerve more than 10%.

In accordance with one embodiment, a method controls bladder function of a patient having a symptom (e.g., related to overactive bladder (OAB) or stress unitary incontinence (SUI)), the symptom related to a symptom signal generated by the patient's body (e.g., an OAB signal). The method provides a neuromodulation device. The neuromodulation device has a main body and a movable arm. A channel is defined by the main body and the movable arm. The channel leads from an exterior of the neuromodulation device to a stimulation chamber having a stimulation electrode therein. The movable arm is transitionable between a closed position and an open position. The movable arm is positioned to the open position. A prescribed somatic motor nerve of the coccygeal plexus and/or the perineal plexus associated with the pelvic floor is positioned within the chamber after positioning the movable arm in the open position. The movable arm is positioned to the closed position after positioning the prescribed somatic motor nerve in the chamber. The method transmits, via the electrode, a symptom control signal (e.g., SUI and/or an OAB control signal) to the prescribed somatic motor nerve. The control signal is configured to activate the pelvic floor in a prescribed manner to mitigate the effect of the symptom signal on the spinal cord.

In some embodiments, the prescribed somatic nerve is positioned within the chamber and contacts the electrode. Positioning the movable arm in the closed position may compress the prescribed somatic nerve in the chamber, slightly deforming it without causing injury. For example, the nerve may be compressed 30% without injury. In some embodiments, the nerve may be compressed 50% without injury. The housing may include a projection configured to press against the movable arm in the closed position. The projection may be configured to limit the amount of compression on the nerve from the movable arm.

In various embodiments, the electrode partially surrounds the nerve. The electrode may contact less than 180 degrees around the circumference of the nerve. The movable arm may be biased towards the closed position. The prescribed somatic motor nerve comprises the perineal nerve, further wherein the perineal or pelvic floor nerve is part of the coccygeal and/or perineal plexus.

In some embodiments, transmitting comprises using a signal generator to produce and transmit the symptom control signal with the electrode. The symptom control signal may be transmitted using one or more of wired or wireless communication media. The symptom control signal may have an amplitude of between about 0.4 milliamps and 4 milliamp. The symptom control signal may be a periodic signal with a plurality of pulses, each pulse having a pulse duration of between about 200 microseconds and 400 microseconds. The symptom control signal may be a periodic signal having a frequency of between about 5 Hertz and 20 Hertz. The symptom control signal may be transmitted for no less than 10 minutes and for no longer than 30 minutes in a single session.

Some embodiments may transmit an SUI control signal to the prescribed somatic motor nerve via the electrode. The SUI control signal may have a frequency that is different from the frequency of the OAB control signal. The SUI control signal may be transmitted after the OAB control signal. The SUI control signal may be configured to strengthen the pelvic floor when applied via the prescribed somatic motor nerve. The SUI control signal may be configured to strengthen or repair the pelvic floor.

In accordance with another embodiment, a system controls bladder function of a patient having a symptom (e.g., related to overactive bladder (OAB) or stress unitary incontinence (SUI)). The symptom is related to a symptom signal generated by the patient's body. The system includes a main body coupled with a movable arm. The main body and the movable arm define a channel therebetween. The channel leads from an exterior of the main body to a stimulation chamber. The movable arm is transitionable between a closed position and an open position. The channel has a gap with a depth that is greater in the open position than in the closed position. The system includes an electrode positioned in the chamber. The electrode is configured to couple with the perineal nerve. The electrode has a receive interface configured to receive an OAB control signal for actuating the perineal nerve. The depth of the gap in the closed position is configured to be less than about 100 micrometers to inhibit the non-destructive passage of the perineal nerve into the chamber. The depth of the gap in the open position is configured to be large enough (e.g., between about 0.5 millimeters and 2.5 millimeters) to allow the nerve (e.g., perineal nerve) to pass.

In some embodiments, the depth of the gap in the closed position is substantially zero. The system may further include a signal generator having a transmit interface configured to communicate with the receive interface of the electrode. The signal generator may be configured to transmit an OAB control signal toward the electrode via the transmit interface and receive interface. The OAB control signal may have a set of prescribed specifications to activate the pelvic floor in a prescribed manner to mitigate the effect of the natural OAB signal on the pelvic floor.

In various embodiments the electrode and signal generator comprise parts of a kit. The signal generator may have memory for storing the set of prescribed specifications. The set of prescribed specifications may include an amplitude of between about 0.4 milliamps and 4 milliamp. The set of prescribed specifications may include the OAB control signal being a periodic signal with a plurality of pulses. Each pulse having a pulse duration of between about 200 microseconds and 400 microseconds. The set of prescribed specifications includes the OAB control signal may be a periodic signal having a frequency of between about 5 Hertz and 20 Hertz. The set of prescribed specifications may include a time frame for transmitting the OAB control signal. The time frame may have a duration of no less than 10 minutes and no more than 30 minutes in a single session.

The signal generator may be configured to transmit an SUI control signal to the perineal nerve. The SUI control signal may have a frequency that is different from the frequency of the OAB control signal. The signal generator may be configured to transmit the SUI control signal after the OAB control signal. The SUI control signal may be configured to strengthen the pelvic floor when applied via the electrode and perineal nerve.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIGS. 1A, 1B, 2A, 2B, 3A, 3B, 4A, 4B, 4C, 5, 6 and 7 schematically show a neuromodulation device configured in accordance with illustrative embodiments. It should be noted that the dimensions listed in these figures are illustrative and not intended to limit various embodiments.

FIG. 8A is an anatomical drawing showing various nerves and relevant anatomy to illustrative embodiments.

FIG. 8B shows the innervation patterns of a target nerve in accordance with illustrative embodiments of the invention.

FIG. 9 is a neuromodulation process in accordance with illustrative embodiments.

FIGS. 10A-10B schematically show the device transitioning from a closed configuration to an open configuration in accordance with illustrative embodiments.

FIGS. 10C-10D schematically show the device transitioning from a closed configuration to an open configuration in accordance with illustrative embodiments.

FIG. 11 schematically shows a close-up view of the arm in accordance with illustrative embodiments of the invention.

FIG. 12 schematically shows a plurality of orientations for the device body and the channel/chamber orientation in accordance with illustrative embodiments of the invention

FIG. 13 schematically shows a device having multiple electrodes and chambers in accordance with illustrative embodiments.

FIG. 14 schematically shows details of the protrusion on the arm in accordance with illustrative embodiments.

FIGS. 15, 16 and 17 schematically show various electrodes in accordance with illustrative embodiments.

FIGS. 18A, 18B, 18C, 18D, 18E, 18F, 18G, 18H, 19A, 19B, 19C, 20A, 20B and 20C schematically show an alternative embodiment of the neuromodulation device in accordance with illustrative embodiments.

FIG. 21 schematically shows a neuromodulation device in accordance with illustrative embodiments.

FIG. 22 schematically shows a neuromodulation device in accordance with illustrative embodiments.

FIGS. 23A, 23B and 23C schematically show a neuromodulation device in accordance with illustrative embodiments.

FIGS. 24A-24B schematically show large diameter nerves positioned within the chamber in accordance with illustrative embodiments.

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

In illustrative embodiments, a nerve is positioned into a stimulation chamber of a neuromodulation device. The device includes a movable arm that opens to permit passage of the nerve into the stimulation chamber. The arm closes to prevent or hinder the nerve from being dislodged from the stimulation chamber without overly compressing the nerve. The movable arm is configured to retain nerves of a variety of sizes within the chamber (e.g., the chamber accommodates nerves of ranges of 0.5 mm -4 mm). In various embodiments, the arm is biased towards a closed or substantially closed position.

Additionally, the device may be configured to retain nerves of different sizes in the chamber and in contact with an electrode while applying an atraumatic retention force on the nerve. The channel similarly is configured to allow passage of the nerve therethrough in response to pressure from the nerve. The necessary pressure to open the channel is configured to be less than a pressure that may damage the nerve. Details of illustrative embodiments are discussed below.

Various embodiments further selectively stimulate the peripheral nerve in areas with a high density of small nerves that uses a combination of lower power stimulation directed at a specific nerve target without a damaging, time intensive and/or traumatic surgical procedure. Prior art electrode arrays disadvantageously do not provide specific enough stimulation to the targeted nerve and can cause unintended side effects.

Similarly, nerve cuffs disadvantageously are limited by pre-defined sizes, which often are suboptimal when coupled to a nerve that is not properly sized. Furthermore, the surgical implantation methods are long, invasive and awkward to do without damaging the nerve, especially as the target nerve temporally decreases in size during implantation. Various embodiments disclosed herein advantageously deliver a selective stimulation signal via electrodes in close contact with a target nerve with a simple implantation procedure.

Furthermore, various embodiments include a neuromodulation device having a channel defined by a plurality of jaws or arms that are movable and/or deformable. The dimensions of the channel are adjustable to allow for entry of nerves of various sizes into the chamber, and also for securing the nerve within the chamber. Details of illustrative embodiments are discussed below.

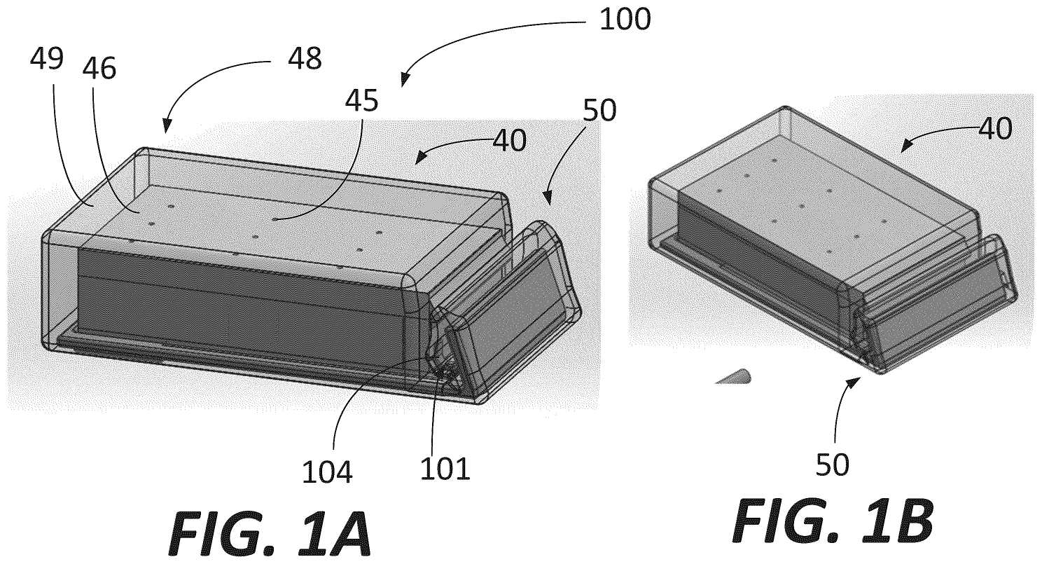

FIGS. 1A-4C schematically show a neuromodulation device 100 configured in accordance with illustrative embodiments. To that end, the neuromodulation device 100 has a main body 40 coupled with a movable arm 50 (also referred to as a movable jaw 50). The arm 50 may be hingedly coupled with the main body 40 (e.g., such that the arm 50 pivots relative to the main body 40).

In various embodiments, the main body 40 may include a housing 48. The housing 48 may also encapsulate at least a portion of the movable arm 50. To that end, the housing 48 may be formed from a resilient or deformable material. The main body 40 and the movable arm 50 (or the portion of the housing 48 surrounding the movable arm 50 and the main body 40) may define a chamber 101 configured to receive a nerve 200 and a channel 102 leading to the chamber 101. In some embodiments, the chamber 101 may be defined by one or more arms 50. The chamber 101 includes at least one electrode 104 to stimulate the nerve 200.

The housing 48 encapsulates a package 46 of the main body 40. The housing 48 includes a buffer layer 49. As known by those of skill in the art, the package 46 encapsulates electronics and semiconductor material within. For example, the package 46 may include different types of circuitry, including stimulator(s), sensor(s), communication(s), and/or power circuitry. In various embodiments, the package 46 may be formed from a substantially rigid material, such as titanium, stainless steel, glass, ceramic, alumina, zirconium, plastic, and/or other generally acceptable hermetic package material. In various embodiments, the electronic package and the nerve/attachment electrode are unitary. In various embodiments, instead of a feedthrough pin extending from the hermetically sealed housing that is welded to the electrode, the feedthrough pin forms the electrode. In such embodiments, the electrode is unwelded (i.e., has no welded joints connecting it to the feedthrough pin). Instead, the feedthrough pin forms the electrode. When the feedthrough pin is embedded in the movable arm (e.g., silicone) it operates as a reinforcement for the deflecting arm.

The package 46 forms a hermetic seal around the internal electronic circuitry. The electronic circuitry is preferably loaded into the hermetic enclosure 46 and sealed in an inert oxygen and water-limited environment. After the hermetic seal is formed, two advantages are provided. First, the hermetic seal ensures that no additional liquid is introduced into the electronic package 46, potentially causing electrical failure. Second, the hermetic seal ensures that the non-biocompatible materials from which the internal electronics are formed do not leach into the body.

Preferably, the package 46 is formed from a glass or ceramic material, which provide reduced interference to radio frequencies used for wireless power and wireless communication relative to other commonly used package 46 materials. The package 46 (also referred to as the hermetic enclosure 46) may be formed from glass wafers directly bonded to one another. Alternatively, the package 46 may be formed from zirconia ceramic with brazed feedthroughs. In various embodiments, the package 46 may be sealed using laser welding titanium surfaces brazed to the zirconia.

The buffer layer 49 covers the package 46 and provides a second biocompatible layer and suitable soft surface within the human body and continuity to portion contacting the nerve 200. As mentioned previously, the arm 50 may include the overmolded housing 46 formed of a softer material (e.g., durometer 30-50 Shore A).

The buffer layer 49 may be formed from a material configured to conform within its resting position in the body. Although the arm 50 and the package 46 may be encapsulated by the housing 46, the buffer layer 49 does not cover the electrodes 104. Accordingly, the housing 48 may have openings for the electrodes 104 and/or an EMG 42. Additionally, vias 45 may be formed through the housing. In various embodiments, the buffer layer 49 can encapsulate between about 20% and about 99% of the device.

Vias 45 may extend through the package 46 and/or the housing 48 (including buffer layer 49). Electrodes 104 and other external electrical connections may be connected to the metalized vias 45. The metalized vias may be formed using platinum feedthrough wires, among other things. The package 46 and the buffer layer 49 may be joined using RTV biocompatible silicone.

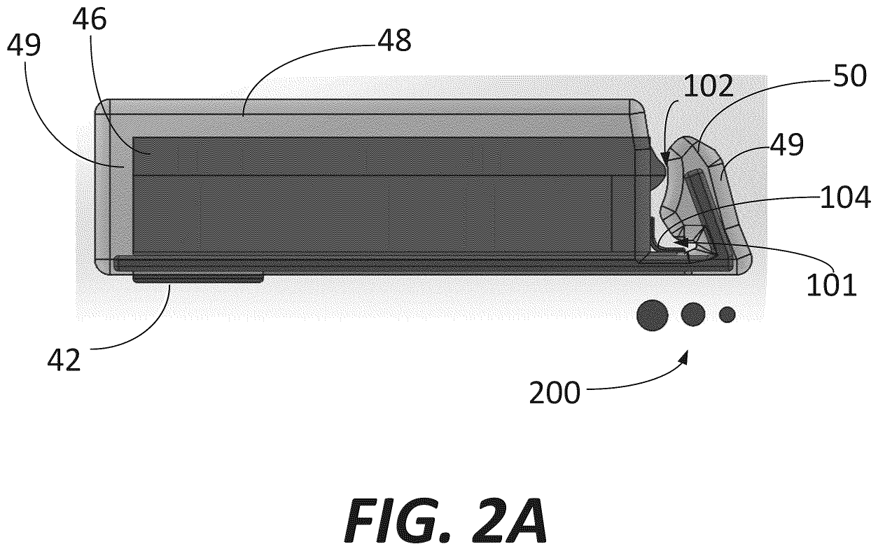

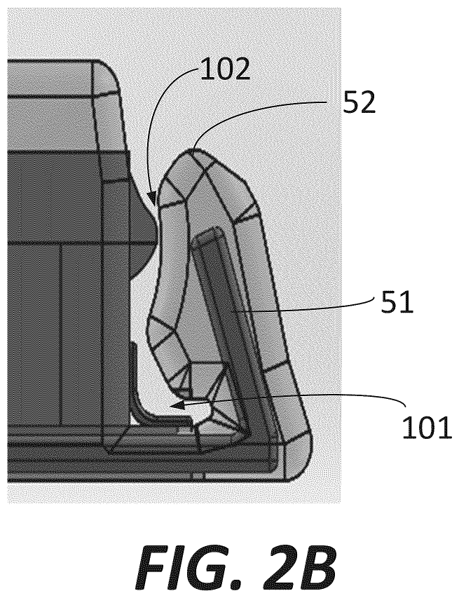

In FIGS. 1A-2B, the arm 50 is depicted in a closed position or a substantially closed position (collectively referred to as the closed position of the device), in which the size of the channel 102 is sufficiently small so that the nerve 200 cannot pass through the channel 102. The arm is movable from the closed position to an open position in which the size of the channel 102 is sufficiently large so that the nerve 200 can pass through the channel 102. In some embodiments, the open position is sufficiently large such that the nerve 200 can be pass through the channel with a reduction in diameter of less than about 30% to about 50% (e.g., by stretching and/or squeezing the nerve through the channel). Various embodiments may be configured to receive a variety of nerve sizes, as discussed further below. In various embodiments, the arm 50 may be biased (e.g., by the use of a biased interior member 51) towards the closed position. A medical practitioner may transition the arm 50 to the open position by applying a force to the arm 50 (e.g., by pulling on a grippable extension 52) or by pressing the arm 50 with the nerve 200 itself. The nerve 200 may then pass through the channel 102 into or out of the chamber 101.

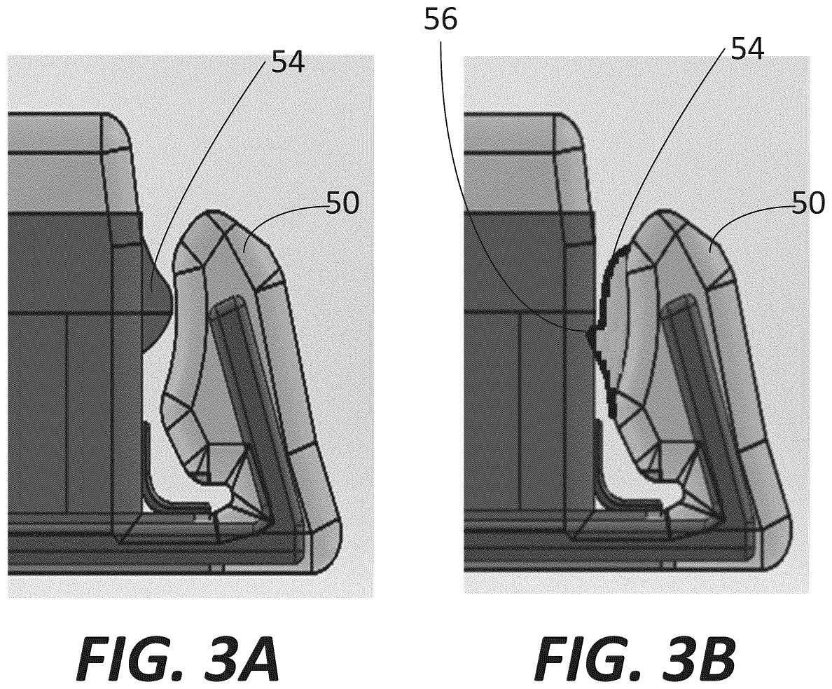

As shown in detail in FIG. 3A, various embodiments may include a protrusion 54 extending from the housing 48. In some embodiments, the protrusion 54 may be formed integrally with the housing 48 and/or the package 46. The channel 102 may be formed, at least in part, between the protrusion 54 and the arm 50. The protrusion 54 may have a convex exterior surface configured to be positioned against a concave surface of the arm 50 (as shown in FIG. 3A). The protrusion 54 and the arm 50 may be sized to prevent the arm 50 from squeezing the nerve 200 in the chamber 101 beyond a pre-set amount.

Although discussion herein may refer to the channel 102 being formed between, or defined by, the arm 50, it should be understood that includes embodiments where the arm 50 is encapsulated by the housing 48. Accordingly, various embodiments are not limited to an uncovered arm 50.

FIG. 3B schematically shows an alternative embodiment without the protrusion 54 extending from the main body (e.g., from the housing 48). Instead, the arm 50 may include the protrusion 54. In a similar manner to FIG. 3A, the protrusion 54 and the housing 48 may be configured to prevent the arm 50 from squeezing the nerve 200 in the chamber 101 beyond a pre-set amount. In various embodiments, to enhance retention of the nerve 200 within the chamber 101, the housing 48 may include a receiving portion 56 (e.g., a recess) configured to receive a portion (e.g., a tip) of the protrusion 54.

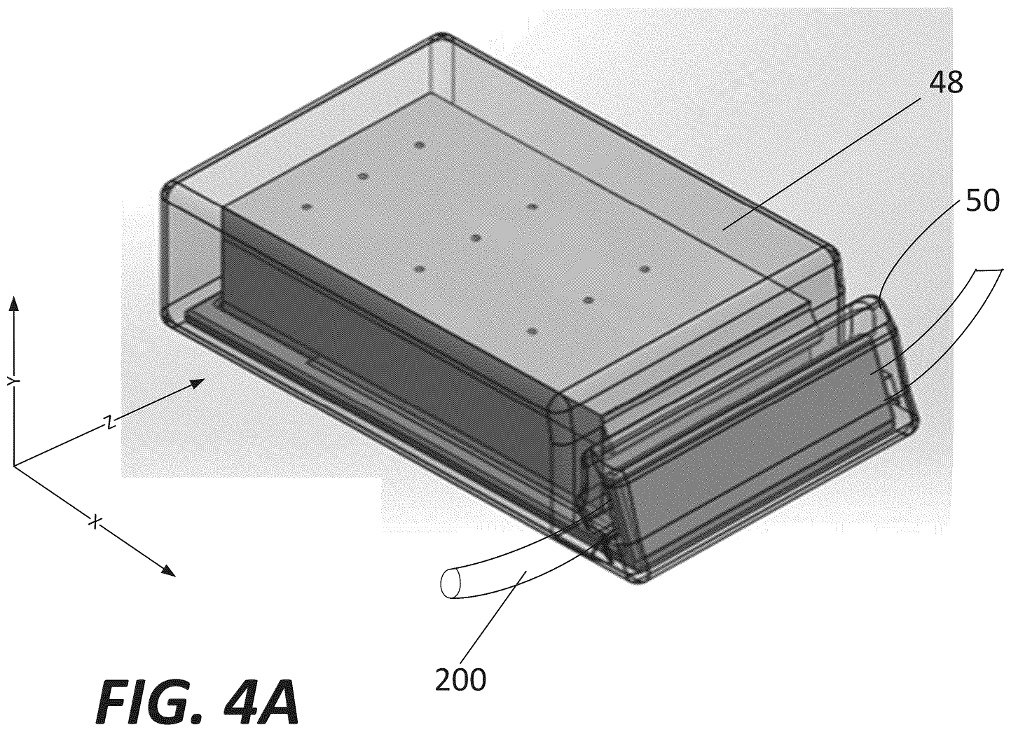

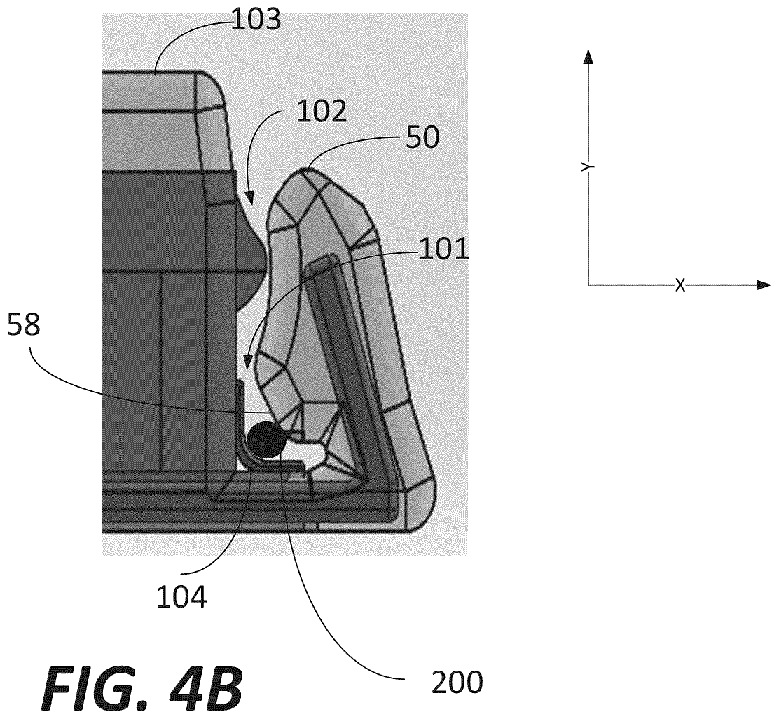

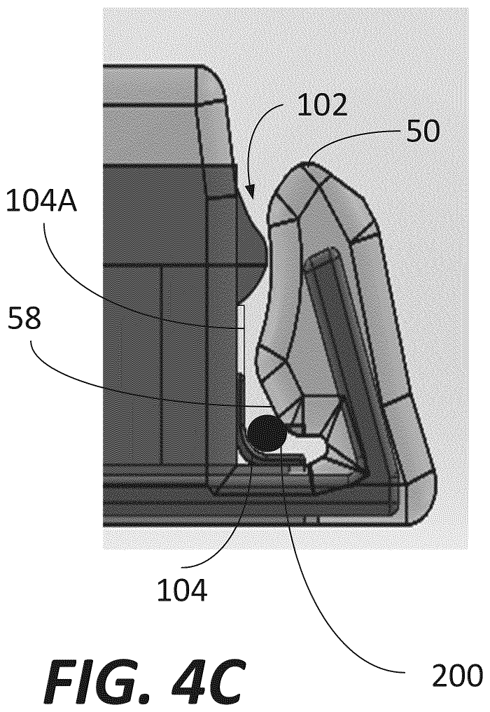

As shown in FIGS. 4A-4B, when the nerve 200 is received in the chamber 101, the nerve 200 is substantially prevented from moving in the X or Y directions (i.e., by the force of the arm 51 pressing the nerve 200). Specifically, the nerve 200 is sandwiched between a nerve contacting surface 58 (which may be concave) and the electrode 104. As shown, the nerve 200 may be slightly compressed. However, in some embodiments, the size of the chamber 101 is such that the nerve 200 may not be compressed at all. In such embodiments, the channel 102 is sufficiently narrow in the closed position such that the nerve 200 cannot escape or enter the channel 102 under normal use. As shown more clearly in FIG. 4A, the nerve 200 is not prevented from moving in the Z-direction (other than by friction that may be caused by moving the nerve 200 through the compressed area). Thus, in various embodiments, the movable arm 50 defines the chamber 101 having a first open end and a second open end when the arm 50 is in the closed configuration. The chamber 101 may also be fluidly coupled with channel 102, which is defined by the movement of the arm 50. The arm 50 may move so that the channel 102 has a gap that is smaller (e.g., substantially zero) than a cross-sectional dimension of the nerve 200 in the closed configuration. The arm 50 may also move so that the channel 102 has a gap that is larger than a cross-sectional dimension of the nerve 200 in the open configuration.

It should be apparent to one skilled in the art that, in contrast to prior art nerve cuffs, the electrodes 104 in various embodiments only partially surround the nerve 200. In other words, the nerve 200 has an outer surface circumference segment that is partially, but not entirely, contacted by the electrode 104.

FIG. 4C schematically shows another embodiment with an extended electrode 104A. In some embodiments, the extended electrode 104A may be a second electrode 104. The extended electrode 104A may stimulate the nerve 104 if as it is positioned in the channel 102. Furthermore, the electrode 104A may be used to stimulate the nerve 200 and to provide information to a practitioner (e.g., that the nerve 200 is not in a desired position to be stimulated within the chamber 101). In various embodiments, the electrode 104A may extend up to and/or onto the protrusion 54. In some embodiments, the chamber 101 can have the function of a recording chamber 101 and/or a stimulating chamber 101. For example, the recording chamber 101 can record electrical activity within the chamber 101 and the stimulating chamber 101 can elicit an electrical stimulus within the chamber 101. To that end, the electrode(s) 104 (and/or electrode 104A) may be used to record signals from and/or to stimulate the nerve 200.

The device 100 may be formed at least in part from a polymer and selected metals. For example, in some embodiments, the neuromodulation device 100 is fabricated using flexible polyimide/Sic substrates with gold metallization in ultra-micro scale using established thin-film and photolithography methods. In another example, the device 100 can be made of SU-8 or other such polymer, using commonly employed microfabrication and photoresist techniques.

As noted above, the device 100 has conductive components. For example, the device 100 can be connected to an electrical pulse generator and/or an electrical stimulator (sometimes generically referred to as a “signal generator”). The device 100, in some cases, can comprise circuitry to communicate with the signal generator. For example, communication circuitry can facilitate magnetic inductive coupling or a direct conductive connection (e.g., via a wire or other hard-wired electrical interface).

The chamber 101 is configured to receive the nerve 200 (e.g., a human somatic motor nerve). The nerve 200 can include multiple nerve axons, a nerve fiber, a nerve bundle, a nerve fascicle, or other similar neuroanatomical structure. It should be understood, however, that the nerve 200 preferably is a functionally intact nerve or a partially-functional nerve. For example, a functionally intact nerve should have a functional pre- and post-synaptic terminal and should be functionally capable of propagating an action potential. For example, the nerve 200, in some embodiments, can have an average diameter of at least 100 μm, at least 200 μm, at least 300 μm, at least 400 μm, at least 500 μm, at least 600 μm, at least 700 μm, at least 800 μm, or at least 900 μm. In some embodiments, the nerve 200 can have an average diameter between about 50 μm and 4 mm, between about 50 μm and 3.5 mm, or some other range between the noted exemplary sizes discussed above.

The chamber 101, in some embodiments, can be generally cylindrical in shape where the ends of a cylindrically shaped chamber 101 are open to permit longitudinal exit of the nerve 200 from the chamber 101 toward the pre- and post-synaptic terminals of the nerve 200. Whereas a cylinder comprises a circular cross-sectional shape, it should be understood that the chamber 101 can also comprise a triangular, square, pentagonal, hexagonal, or polygonal cross-sectional shape having n number of sides, while maintaining a general 3-dimensional structure resembling a cylinder, or a pipe, having open ends and operable to receive the nerve 200. Some embodiments of the chamber 101 have no specific cross-sectional shape and instead may be irregularly cross-sectionally shaped.

The chamber 101, in some embodiments, can be in fluid communication with an external surface 103 and a second (opposite) external surface 103 of the device 100. Specifically, the first and second external surfaces 103 in this example are on opposite sides of the device 100 and the chamber 101, positioned between the first and second external surfaces and in fluid communication with each opposing first and second external surfaces 103 of the device 100.

As a three-dimensional region, the chamber 101 has a length, depth, and a height. The length of the chamber 101 corresponds to a z-axis that traverses longitudinally along the nerve 200 extending through the device 100. The depth of the chamber 101 corresponds to an x-axis, while the height corresponds to the y-axis. In some embodiments, the chamber 101 can have an average length of at least 50 μm, at least 100 μm, at least 500 μm, or at least 1000 μm. In some embodiments, the chamber 101 can have an average length of between about 50 μm and 11 mm (e.g., about 2 mm).

In some embodiments, the chamber 101 can have an average length of between about 10 μm and 5 mm, between about 10 μm and 3 mm, between about 10 μm and 1 mm, between about 50 μm and 2 mm, between about 50 μm and 11 mm, or between about 10 μm and 11 mm.

Various dimensions of the chamber 101 are described below. It should be assumed that the dimensions of the chamber 101 are when the chamber is in the closed position. As described previously, the chamber 101 is partially defined by the arm 50, which is movable. Thus, the chamber 101 has a closed configuration (also referred to as a closed position) and an open configuration (also referred to as an open position). The chamber also has intermediary configurations between the open and closed configuration, but the terminal configurations are described herein. It should be assumed that the dimensions of the chamber 101 described herein refer to the closed configuration, unless the context otherwise requires.

As noted, the depth and height of the chamber 101 can be considered to correspond to cross-sectional dimensions of an x-y-plane orthogonal to the z-axis of the chamber 101. For example, a cylindrically shaped chamber 101 can have a depth and height corresponding to a diameter of the chamber 101. The diameter of a non-cylindrically shaped chamber 101 can be measured by averaging the distance of measurements intersecting the center point of a cross-section of the chamber 101, wherein the center point is positioned on the z-axis extending through the middle of the chamber 101. In some embodiments, the chamber 101 can have an average diameter of about less than 10 mm. In some embodiments, the chamber 101 has an average diameter of about 10 μm (micrometers) to about 2000 μm, about 10 μm to about 4000 μm, 10 μm to about 3000 μm, about 1 μm to 2000 μm, about 10 μm to 1000 μm, about 10 μm to 900 μm, or about 10 to 800 μm, or about 10 μm to 500 μm.

In some embodiments, the chamber 101 has an average diameter that is substantially the same or ten percent smaller than the average diameter of a target nerve 200. In some embodiments, the average diameter of the chamber 101 is no more than 5 percent larger or no more than 5 percent smaller than the average diameter of the target nerve 200. In some embodiments, the average diameter of the chamber 101 is no more than 15 percent larger or no more than 15 percent smaller than the average diameter of the target nerve 200. For example, for a target nerve 200 having an average diameter of about 80 μm, the device 100 can have an average diameter of no less than about 56 μm, and no more than 104 μm. In some embodiments, the chamber 101 has an average diameter that is about 80-120 percent of a target nerve 200, about 85-115 percent of a target nerve 200, about 90-110 percent of a target nerve 200, about 95-105 percent of a target nerve 200, or about 100 percent or equal in size of a target nerve 200.

In some embodiments, the channel 102 is defined by two walls that can provide upper boundary and lower channel boundaries. In some instances, the distal end of the channel 102 can be in fluid communication with the interior of the chamber 101 and the proximal end of the channel 102 can be in fluid communication with the exterior surface 103 of the device 100. Thus, the distal end of the channel 102 is open to the chamber 101. In some embodiments, the chamber 101 is indefinitely or constantly open to the channel 102, such that the distal opening of the channel 102 into the chamber 101 does not close. Moreover, the channel 102 can connect the interior of the chamber 101 to the external surface 103 of the device 100. Thus, in this embodiment, the chamber 101 is essentially in constant communication with an exterior surface 103 of the device via the channel 102. For example, the chamber 101 remains open to the channel at all times and the channel 102 remains open to an exterior surface at all times.

Various dimensions of the channel 102 are described below. It should be assumed that the dimensions of the channel 102 refer to when the channel 102 is in the closed position. As described previously, the channel 102 is partially defined by the arm 50, which is movable. Thus, the channel 102 has a closed configuration (also referred to as a closed position) and an open configuration (also referred to as an open position). The channel 102 also has intermediary configuration between the open and closed configuration, but the terminal configurations are described herein. It should be assumed that the dimensions of the channel 102 described herein refer to the open configuration, unless the context otherwise requires. This is in contrast to discussion of the chamber 101, which in general refers to dimensions of the chamber in the closed configuration.

As a three-dimensional region, the channel 102 has a length, a height, and a width (also referred to as a gap or an opening). Similar to the length of the chamber 101 described above, a length of the channel 102 corresponds to a measurement along a z-axis, which traverses longitudinally along the nerve 200. The length can be measured at any point along the channel 102 between the distal end of channel opening into the chamber 101 and the proximal end of a channel opening to an exterior surface 103 of the device. In some embodiments, the distal end of the channel 102 can be in fluid communication with the chamber 101 for the entire length of the chamber 101. In some cases, the average length of the channel 102 is substantially the same as the average length of the chamber 101 of the device 100 described herein. In various embodiments, the length of the channel 102 and/or the chamber 101 does not change from the open position to the closed position.

In some embodiments, the channel 102 can have an average length of at least 100 μm. In some embodiments, the channel 102 can have an average length of at least 1000 μm, at least 2000 μm, or at least 4000 μm, or at least 6000 μm. In some embodiments, the channel 102 can have an average length of between about 100 μm and 10 mm. In some embodiments, the channel 102 can have an average length of between about 100 μm and 6 mm, between about 100 μm and 3 mm, between about 100 μm and 8 mm, or between about 10 μm and 9 mm (e.g., an embodiment of the device with a battery).

The height of the channel 102 corresponds to a distance measured between the distal opening and the proximal opening of the channel 102, wherein the distance is measured along an imaginary centerline positioned equidistant between each channel wall. In some cases, the height can be a linear measurement. For example, in some cases, the channel 102 is a linear channel 102. In other cases, the channel 102 can be non-linear, wherein a non-linear channel comprises one or more turns, curves, or bends in the channel walls (e.g., from protrusion 54). Thus, in some instances, the height of a non-linear channel 102 can be measured by measuring the distance along the imaginary centerline of the channel 102 between the distal opening and proximal opening of the channel 102, and along each bend in the non-linear channel 102. For example, in some embodiments, the channel 102 can comprise an “L” shape, such that the channel 102 height measurement comprises a 90-degree turn and each end of the “L” corresponds to the distal and proximal openings of the channel. In an exemplary channel having a 90-degree turn, the height can be measured by summing the distance of an imaginary centerline of the channel for each arm in the “L” of the channel 102 extending between the proximal opening and the distal opening of the channel 102 to where the imaginary lines of each arm meet. The shape of the channels can also include other configurations such as T, Z and S, and others.

In some embodiments, the channel 102 can have an average height of between about 100 μm and 5 mm (e.g., 4.5 mm). In some embodiments the channel 102 can have an average height of between about 100 μm and 5 mm, between about 100 μm and 1 mm, between about 100 μm and 2 mm, between about 100 μm and 3 mm, between about 100 μm and 4 mm, or between about 100 μm and 5 mm.

The width of the channel 102 corresponds to a measurement of the channel 102 positioned in an x-y plane that is orthogonal to the z-axis, as described above. The width of the channel 102 can be constant, such that the width of the channel 102 does not change between the proximal opening and distal opening of the channel 102. That is, in some embodiments, the width of the channel 102 comprises less than 10 percent variability of an average width across an entire height of the channel 102. In some cases, the channel 102 comprises less than 5 percent variability, less than 3 percent variability, or less than 2 percent variability of an average width along an entire height measurement of the channel 102. In some cases, a width can be determined by measuring the shortest distance between the main body 40 (e.g., the housing 48) and the arm 50.

The width of the channel 102, in some embodiments, is less than the diameter of a target nerve 200. For example, in some embodiments, the channel 102 width can be at least 5 percent smaller than a diameter of a target nerve 200. In some embodiments, the channel 102 width can be at least 5 percent, at least 10 percent, at least 15 percent, at least 20 percent, at least 25 percent, at least 30 percent, at least 35 percent, at least 40 percent, at least 45 percent, or at least 50 percent smaller than a diameter of the nerve 200. In some embodiments, the channel 102 width can be no more than 60 percent smaller than a target nerve 200 diameter. In other embodiments, the channel 102 width can be no more than 50 percent smaller than a target nerve 200 diameter. In some embodiments, the channel 102 width can be between about 5 percent and 60 percent smaller than a diameter of a target nerve 200. In some cases, the channel 102 width can be between about 10 percent and 50 percent, between about 10 percent and 40 percent, between about 15 percent and 40 percent, between about 20 percent and 35 percent, or between about 20 percent and 40 percent smaller than a diameter of a target nerve 200.

Furthermore, similar to the chamber 101 described above, the channel 102 can have open ends in fluid communication with the open ends of the chamber 101 such that the nerve 200 can be inserted into the chamber 101 by sliding, moving, or inserting a longitudinal section of the nerve 200 into the chamber 101 via the channel 102 when the channel 102 is in the open position. Thus, the channel 102 is configured to receive the target nerve 200. Moreover, in some embodiments, the channel 102 can be in fluid communication with one, two, or all three external surfaces 103 of the device 100. For example, the channel 102 can be open to an interior of the chamber 101 at the distal end of the channel and the channel can extend along the height of the channel to the third external surface 103 at the proximal end of the channel, while maintaining fluid communication with the first external surface 103 and the second external surface 103 on opposing sides of the device 100 corresponding to opposing ends of the z-axis.

As noted above, the device 100 has at least one electrode 104 within the chamber 101. The electrode 104 can include various types of electrodes, including, among other types, fiber or flat electrodes, thin film electrodes, or needle electrodes. As an example, the electrode 104 can be implemented as a thin film electrode with a recording or stimulating surface within 100-2000 μm2 or within 50 μm of an outer surface of the nerve 200 when coupled within the chamber 101. A needle electrode in the chamber can have a needle shaped recording and/or stimulating surface that can penetrate the surface of the nerve 200 when in the chamber 101. The electrode that penetrates a nerve in the chamber 101 can stimulate and/or record intraneurally, which can provide greater selectivity and/or resolution when recording and/or stimulating. Additionally, the electrode 104 can be positioned on any of a number of other surfaces within the chamber 101, including the top, a bottom, and/or side chamber surfaces.

A combination of electrode types can be used when the chamber 101 has more than one electrode 104. For example, both flat electrodes and/or needle electrodes can be used in a recording and/or stimulating chamber 101. Among others, the electrode(s) 104 can be mono-polar, bi-polar, tri-polar, or a multi-electrode array electrodes. In some cases, a plurality of electrodes 104 can be configured in a tripolar configuration, which should provide improved nerve specificity and/or selectivity while simultaneously reducing extraneous biological noise.

As noted above, the electrode 104 can be formed at least in part from one or more conductive metals. For example, the electrode can be formed at least in part from gold, titanium nitride (TiN), iridium oxide (IrO), iridium, carbon nanotubes, graphene, graphene oxide, and/or platinum (Pt). The electrode 104, in some instances, can have a charge injection capacity of about 0.1 mC/cm2 or greater. Further, in some embodiments, the electrode 104 can be a wired or a wireless electrode. A wireless electrode 104 can have a wireless integrated circuit that enables communication with external devices.

In some embodiments, the electrode 104 can have a stimulating and/or recording surface area of between about 25 μm² and 25 mm². In some instances, the electrode comprises a stimulating and/or recording surface area of between about 100 μm² and 1 mm² or between about 100 μm² and 0.5 mm².

Illustrative embodiments use prescribed neuromodulating techniques to selective activate one or more pelvic floor muscles. Among others, those pelvic floor muscles may include the cremaster muscle, bulboglandularis muscle (Bgm), ischiocavernosus muscle (Ism), bulbospongiosus muscle (Bsm), pubococcygeus muscle (Pcm), iliococcygeus muscle (Icm), coccygeus muscle (Cgm), or puborectalis muscle (Prm). Preferably, however, the pubococcygeus muscle is actuated using the perineal nerve. Alternatively, it can also be actuated using the pubococcygeal branch. In some embodiments, the pelvic floor muscle can be neuromodulated or stimulated simultaneously or independently of one or more other pelvic floor muscles. In some instances, a pelvic nerve can include any nerve, nerve bundle, nerve fascicle, or nerve tract that innervates a pelvic floor muscle or pelvic organ, including the pudendal nerve, the clitoralis nerve or the dorsal nerve of the penis, or any branch of these or other nerves in the pelvis. In various embodiments, some embodiments may be used to help treat pelvic organ prolapse by stimulation the distal terminal branches of the pudendal nerve (e.g., perineal, inferior rectal) and/or terminal branches of the levator ani nerve. Furthermore, some embodiments may be used in veterinary applications to stimulate animal nerves.

It should be understood than the device 100 discussed above is but one example of a neuromodulation device that can be used to stimulate the appropriate somatic nerve. For example, two or more devices 100 can be used to stimulate two or more pelvic floor muscles or organs. Furthermore, since the device 100 can record and/or stimulate, two or more devices can be used on the same pelvic floor muscle to independently record and stimulate, or one device can be used to record and stimulate the pelvic floor muscle or organ.

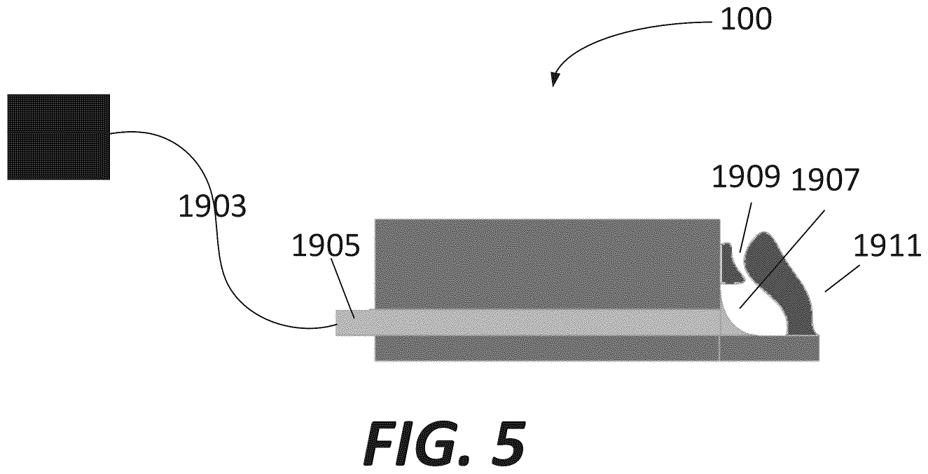

FIG. 5 schematically shows another embodiment of the device 100 in accordance with illustrative embodiments. Note that this figure has different reference numbers for the same components from those above, but similar components have the same or similar functionality. As shown, the device includes a battery or otherwise powered pulse generator and electronic controller 1901 connected to an electrode 1905 by way of a conductive wire 1903. As illustrated, the electrode is a part of the chamber 1907 of the neuromodulation device 1911. As illustrated, the neuromodulation device 1911 may include a curved channel 1909 configured to receive a nerve. In some embodiments, at least a portion of the width of the channel 1901 may be smaller than the diameter of the target nerve in the closed configuration and/or the open configuration. Accordingly, the target nerve may be temporarily be reversibly compressed or stretched, and slid through the channel until the target nerve is held within the chamber 1907. In some embodiments, the chamber 1907 may have a diameter greater than the nerve, such that the nerve is not compressed or stretched within the chamber 1907. Alternatively, the chamber 1907 may have a diameter smaller than the nerve. Accordingly, in such an embodiment, at least a portion of the nerve may extend into the channel 1909 while a substantial portion of the nerve is contained within the chamber 1907. Further, the chamber 1907 may provide an isolated fluidic environment that allows for the targeted and specific stimulation of the portion of the nerve held within the chamber 1907.

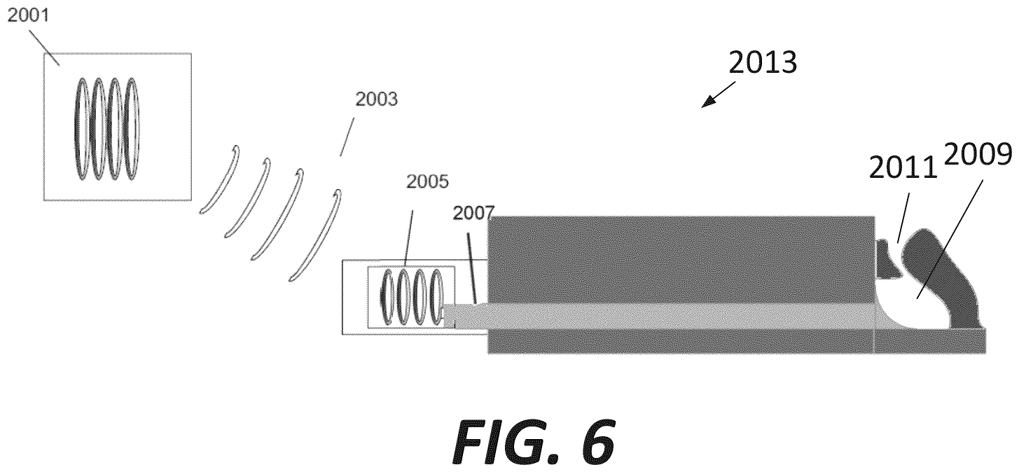

FIG. 6 schematically shows a wireless neuromodulation device and external system in accordance with illustrative embodiments. As with FIG. 5, this figure has different reference numbers for the same components from those above, but similar components have the same or similar functionality. The device has an external battery or otherwise powered pulse generator and electronic controller 2001 that includes a coil to transmit power, data and/or control signals to a wireless neuromodulation device 2013. In particular, an electromagnetic field 2003 may couple the pulse generator and electronic controller 2001 to the wireless neuromodulation device 2013. Corresponding electronics and a magnetic induction coil 2005 in the neuromodulation device 2013 may be connected to the conductive material used as electrode(s) 2007. The electrodes 2007 may form part of the chamber 2009. As discussed with other embodiments, the neuromodulation device of this figure may include a chamber 2009 configured to receive the nerve, and a channel 2011 (e.g., an expandable gap) through which the nerve may pass as it is placed in the chamber 2009.

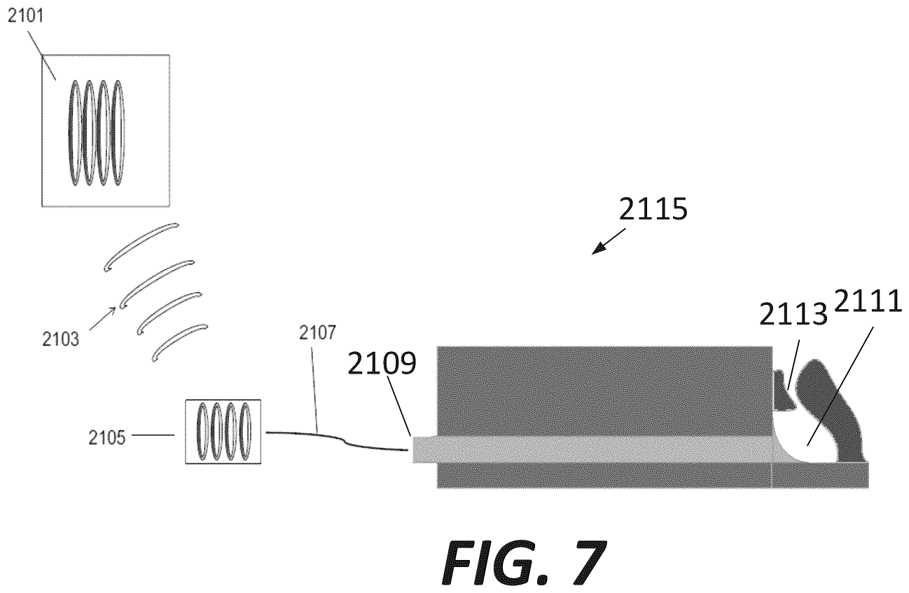

FIG. 7 schematically shows another wireless embodiment of the system. As with FIGS. 5-7, this figure has different reference numbers for the same components from those above, but similar components have the same or similar functionality. To that end, an external battery powered pulse generator and electronic controller 2101 with a coil is configured to transmit power, data and/or control signals to a neuromodulation device 2115. An electromagnetic field 2103 couples with the coil 2105 of the neuromodulation device 2115. In some embodiments, the electronics and coil 2105 may be spaced apart and implanted separately from the stimulating elements of the neuromodulation device 2115. The location of the electronics and coil 2105 may be chosen to optimize the signal strength and quality of transmissions between the external pulse generator and electronic controller 2101 and the neuromodulation device 2115.

Further, the separate electronics and coil 2105 configuration may lead to reduced battery requirements and applied voltage and/or amplitude. A conductive wire 2107 may couple the electronics and coil 2105 to the neuromodulation device 2115, and more particularly to the electrodes 2109 which may be located within a chamber 2111. As with some other embodiments, the neuromodulation device 2115 may include a chamber 2111 configured to receive the nerve, and a channel 2113 (or expandable gap) through which the nerve may pass as it is placed in the chamber 2111.

Among other benefits, connecting implanted electronics with a receiver coil to the neuromodulation device effectively separates the receiver coil. This enables a medical practitioner to implant the receiver coil in the same location and orientation relative to the hard tissue structures of the body regardless of where the electrode and neuromodulation device is positioned. This consistency in the location and orientation of the receiver coil is expected to provide a more consistent, efficient and reliable coupling and induction in the implanted coil and electronics. This may help avoid variability in stimulation or variability in external coil position or orientation requirements.

Based on their experimentation, the inventors discovered that stimulation of pelvic floor muscles can activate a type of sensory fiber not previously associated with urinary incontinence. Specifically, this includes the proprioceptive type Ia and Ib large myelinated sensory afferents. Using the appropriate parameters/specifications, these sensory afferents can then be targeted for selective neuromodulation to activate a previously unknown sensory pathway to the central nervous system, including pathways in the spinal cord and brainstem involved in store/voiding control.

To those ends, the inventors were surprised to learn that acute wireless electrical stimulation of the bulbospongiosus nerve in certain animals increased the maximal urethral pressure and voiding efficiency significantly, proportional to the stimulation frequency. Acute stimulation was sufficient to induce a 3-fold increase in urethral pressure and voiding volume, indicating that activation of this perineal nerve strengthens the urethral sphincter supporting continence and voiding efficiency. This result confirmed the ability of miniature wireless stimulators for effective pelvic floor muscle contraction, and as a strategy to reverse SUI-like features in aging and multiparous rabbits.

The coccygeal nerve plexus is anatomically and functionally different compared to the pelvic nerve plexus, which includes the pudendal nerve. Therefore, sacral neuromodulation (SNM), percutaneous tibial nerve stimulation (PTNS), and pudendal nerve stimulation (PNS), all part of the pelvic plexus, primarily target the same sacral (S2-3) levels in the spinal cord, where axons in the sacral or peripheral nerves are mixed anatomically and functionally with pain, mechanoception motor, proprioceptive and autonomic neurons. The inventors believe, but have not confirmed, that the mechanism of action of SNM, PTNS, and PNS for the treatment or urge incontinence involves the modulation of the mechanosensory afferent fibers.

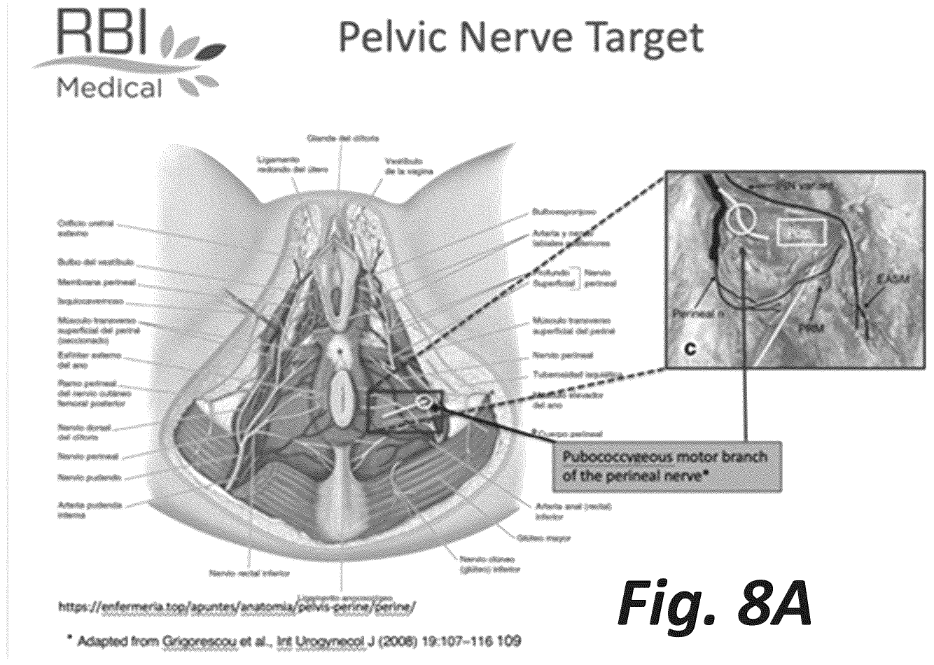

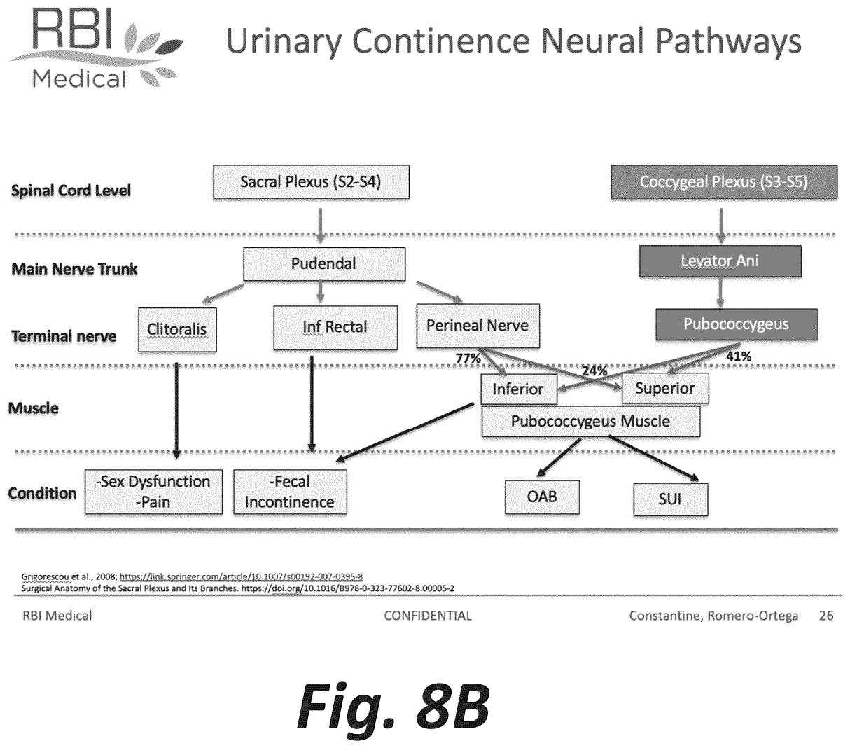

In contrast, the inventors found that the axons innervating the pelvic floor muscles are innervated by either the pelvic or coccygeal plexus, and are more uniform anatomically and functionally, and composed mostly from motor somatic efferent and proprioceptive (Ia/Ib) sensory afferents, which enter the spinal cord through anterior (S2-3) and/or a more posterior (S4-5) spinal cord levels, through the perineal and or levator ani nerves; respectively. Yet, the proprioceptive axons ascend all the way to lumbar (L)7 connecting the function of the PFMs to those of the bladder and other pelvic organs. The inventors thus discovered that stimulation of the proprioceptive sensory afferents also inhibits the activity from the bladder and thus, discovered that such stimulation can be a useful treatment for overactive bladder syndrome. Illustrative embodiments have selected the motor branch of the perineal nerve, which controls the pubococcygeous muscle. See FIG. 8A for details.

FIG. 8B shows the innervation patterns of a target nerve in accordance with illustrative embodiments of the invention. As shown, the pubococcygeus muscle is innervated by branches of the perineal and coccygeal plexus at different ends. While the pelvic nerve is a branch of the perineal plexus innervating the anterior Pcm, it also receives innervation of the pubococcygeus nerve, which branch from the levator ani. The percentage innervations are based on the two references listed in the slide.

Various embodiments may couple and stimulate nerves to treat, among other things: female sexual dysfunction, male erectile dysfunction, fecal incontinence including but not limited to stimulation of the sacral nerve, overactive bladder/urge incontinence including but not limited to stimulation of the sacral nerve or tibial nerve stimulation, pain management including chronic pelvic pain, headaches, somatic (i.e. muscle, limbs) and visceral either by activating mechanoreceptive or proprioceptive fibers that compete with nocioceptive signals or by directly blocking the signals of pain fibers, blood pressure management, sleep apnea, stimulation for nausea, stimulation for tremors, and/or stimulation of the vagus nerve, sacral roots and ventral roots for various applications. Various embodiments may stimulate various nerves in accordance with the stimulation parameters provided herein (e.g., for OAB and SUI) and for other treatments in accordance with the same or different parameters (e.g., as those used for OAB and/or SUI).

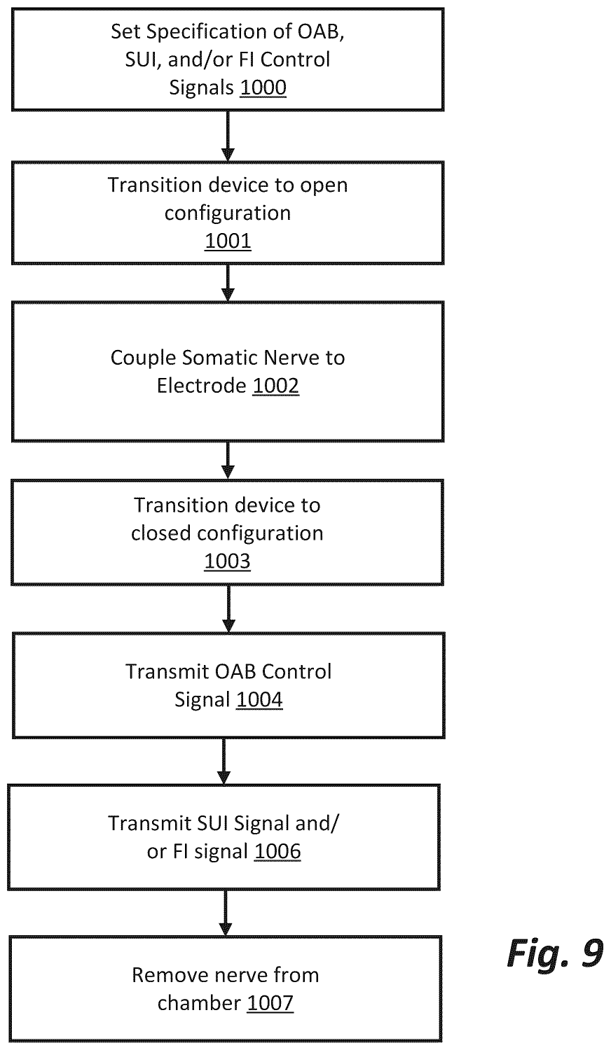

FIG. 9 shows a neuromodulation process in accordance with illustrative embodiments. It should be noted that this process is simplified from a longer process that normally would be used. Accordingly, the process likely has many steps that those skilled in the art likely would use. In addition, some of the steps may be performed in a different order than that shown. Additionally, or alternatively, some of the steps may be performed at the same time. Those skilled in the art therefore can modify the process as appropriate.

The process uses at least one “control signal” to neuromodulate the appropriate motor and proprioceptive nerve branches from either the coccygeal and/or perineal plexus, which, in this example, is the perineal nerve. Those control signals are referred to as:
1) An “OAB control signal” to directed toward managing overactive bladder syndrome, and
2) An “SUI control signal” directed toward managing stress urinary incontinence.

The process of FIG. 9 thus begins at step 1000, which sets the specifications of the control signals. In various embodiments, the control signals may be OAB and/or SI control signals. These specifications may be stored in a database, memory, or other storage medium for use in subsequent steps. Among other locations, such a location may be part of the device 100 itself, part of the signal generator, some somewhere across a local area network (e.g., an enterprise network), or somewhere across a larger wide area network (e.g., the Internet). The inventors determined, using calculations and experimentation, that the succeeding specifications should produce satisfactory results.

The process uses at least one “control signal” to neuromodulate the appropriate nerve, e.g., a coccygeal plexus somatic nerve, which, in this example, may be the perineal nerve. Those control signals are referred to as:
1) The “OAB control signal”—stimulation parameters directed toward managing overactive bladder syndrome, and
2) The “SUI control signal”—stimulation parameters directed toward managing stress urinary incontinence.

Specifically, the OAB control signal may have a set of one or more of the following specifications for humans:
an amplitude of between about 0.4 milliamps and 1 milliamp,
each pulse having a pulse duration of between about 200 microseconds and 400 microseconds (when the OAB control signal is a periodic signal),
a frequency of between about 5 Hertz and 20 Hertz (when the OAB control signal is a periodic signal), and
duty cycle of between about 5% and 100% (e.g., more specifically between about 8% and 12%).
a stimulation pulse of about 10-20 seconds followed by about 2.5 minutes off, repeated at least 3-4 times within a single session
a duration for transmitting the OAB control signal of no less than 10 minutes and for no longer than 30 minutes in a single session.

These values can be scaled appropriately for different mammals. In addition, these values can be adjusted as a function of the number of treatments per day or week, the individual, and/or the disease severity.

The SUI control signal may have a set of one or more of the following specifications for humans:
an amplitude of between about 0.5 milliamps and 2 milliamp,
each pulse having a pulse duration of between about 200 microseconds and 400 microseconds (e.g., when the OAB control signal is a periodic signal),
a frequency of between about 60 Hertz and 100 Hertz (when the OAB control signal is a periodic signal), and
duty cycle of between about 5% and 100% (e.g., more specifically between about 8% and 12%).
a stimulation pulse of about 10-20 seconds followed by about 2.5 minutes off, repeated at least 3-4 times within a single session a duration for transmitting the SUI control signal of no less than 30
seconds and for no longer than 120 seconds in a single session.

As another example, the OAB control signal may have a set of one or more of the following specifications for humans:
an amplitude of between about 0.4 milliamps and 1 milliamp,
each pulse having a pulse duration of between about 200 microseconds and 400 microseconds (when the OAB control signal is a periodic signal),
a frequency of between about 5 Hertz and 20 Hertz (when the OAB control signal is a periodic signal), and
a duration for transmitting the OAB control signal of no less than 10 minutes and for no longer than 30 minutes in a single session.

These values can be scaled appropriately for different mammals. In addition, these values can be adjusted as a function of the number of treatments per day or week, the individual, and/or the disease severity.

The SUI control signal may have a set of one or more of the following specifications for humans:
an amplitude of between about 0.4 milliamps and 4 milliamp,
each pulse having a pulse duration of between about 200 microseconds and 400 microseconds (e.g., when the OAB control signal is a periodic signal),
a frequency of between about 60 Hertz and 100 Hertz (e.g., when the OAB control signal is a periodic signal), and
a duration for transmitting the SUI control signal of no less than 30 seconds and for no longer than 120 seconds in a single session.

As with the specifications for the OAB control signal, these values can be scaled appropriately for different mammals. In addition, these values can be adjusted as a function of the number of treatments per day or week, the individual, and/or the disease severity.

Generically, a symptom control signal for neuromodulation of the pelvic floor may have a frequency of about 2 Hz to about 100 Hz. A nerve block signal may use higher frequencies, such as about 1 kHz to about 40 kHz. However, some embodiments may use low frequencies (e.g., <10 Hz for nerve blocking).

Advantageously, both afferent and efferent control signals may be transmitted on the same nerve using the same neuromodulation device 100.

TABLE 2
TREATMENT PARAMETERS FOR FECAL INCONTINENCE
(FI) AND OAB IN HUMANS

| | Target | | | Pulse | Duty | Treatment |
|---|---|---|---|---|---|---|
| | Nerve | Frequency | Amplitude | Duration | Cycle | Duration |
| FI | Inferior | 2 Hz- | Sub- | 200 | (a) 15 | About 10 |
| (afferent | Rectal | 20 Hz | threshold. | microsec.- | min | min.- |
| control | Nerve | | Bi-Phasic | 300 | continuous | about 30 |
| signal) | | | pulse. | microsec. | to | min. |
| and/or | | | 0.4 mA to | | (b) 15 | 1-3 × a day. |
| OAB | | | 1.0 mA. | | sec | |
| (afferent | | | | | ON, 2.5 | |
| control | | | | | min | |
| signal | | | | | OFF, | |
| | | | | | repeating | |
| | | | | | 4 | |
| | | | | | times | |
| FI | Inferior | 50 Hz- | Threshold | 250 | 15 sec | About 10 |
| (efferent | Rectal | 80 Hz | Bi-Phasic | microsec.- | ON, 2.5 | min.- |
| control | Nerve | | pulse. | 400 | min | about 13 |
| signal) | | | 0.5 mA to | microsec. | OFF, | min. |
| | | | 2.0 mA | | repeating | 1-3 × a day. |
| | | | | | 4 | |
| | | | | | times. | |

Experimentation by the inventors has shown that efferent stimulation of the inferior rectal nerve using the above referenced stimulation parameters may close the external, and secondary rectal sphincter.

Details of various control signals that may be used are described in co-pending U.S. patent application Ser. No. 17/985,843, which is incorporated herein by reference in its entirety.

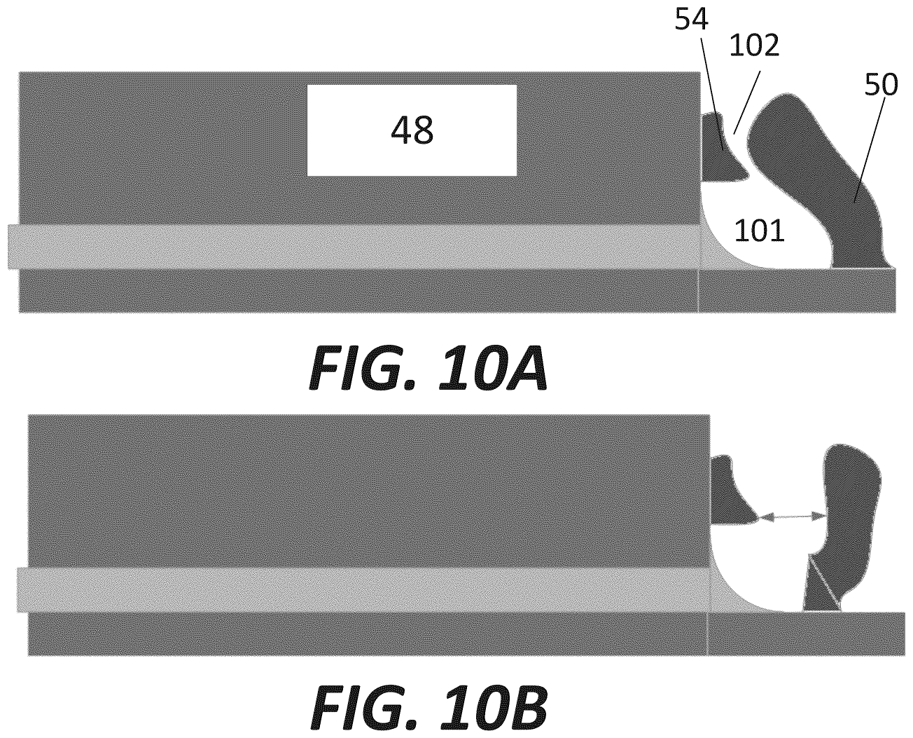

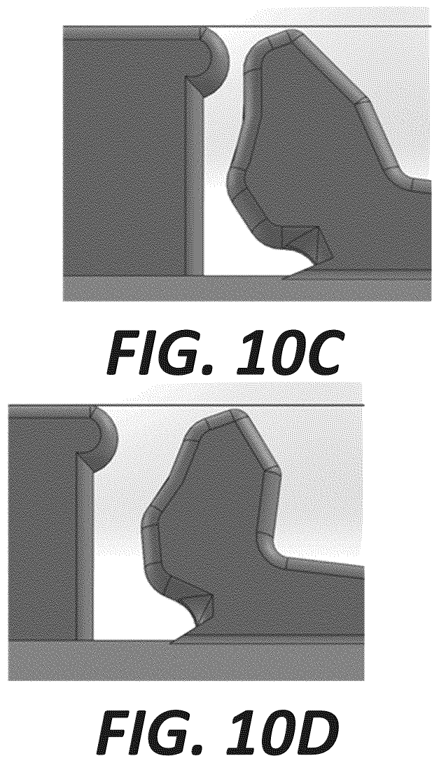

The process continues to step 1001, which transitions the device 100 to an open configuration. As described previously, the device 100 has an open configuration and a closed configuration. FIGS. 10A and 10C schematically show various embodiments of the device 100 in a closed configuration. In the closed configuration, a depth of the channel 102 (also referred to as a gap) between the movable arm 50 and the housing 48 (or projection 54) is sufficiently small so that the target nerve cannot pass through the channel 102 into the chamber 101 without damage. Thus, transitioning to the open configuration allows the nerve to pass into the chamber.

FIGS. 10B and 10D schematically show various embodiments of the device 100 in the open configuration. In various embodiments, the opening of the channel may have a tapered or “V” shape to help guide the nerve into the chamber. In the open configuration, the depth of the channel 102 is large enough so that the nerve can pass through the channel 102 into the chamber 101. It should be understood that the open configuration may be, but does not necessarily have to be, the maximum open position. Meaning, the device may be in the open configuration even if the arm 50 may be moved additionally to make the gap larger. In various embodiments, the size of the gap may be determined by measuring the shortest distance between the housing 48 and the arm 50.

In various embodiments, the size of the gap in the closed configuration is equal to or less than the diameter of a target nerve 200. For example, the dimension of the closed gap may be configured to be 30-50% smaller than the target nerve 200. Various embodiments may have a gap in the closed configuration of about 0 mm to about 0.7 mm, for example 0.5 mm. Various embodiments may have a gap in the open configuration of about 0.75 mm, 1.75 mm, or 2.75 mm.

Transitioning to the open configuration may be accomplished by a medical practitioner pulling the movable arm 50 to cause a gap and/or to increase the size of the gap sufficiently to allow the nerve to pass through (or a stretch nerve to pass through), pressing a button, or using the nerve itself to open the channel. In some other embodiments, the arm 50 may be electromechanically operated (e.g., via the push of a button). In some other embodiments, the biased arm 50 (e.g., polymeric “spring loading” in its elastic range, elastomeric properties of the material itself, addition of spring, etc.) may be pre-opened or may open using a delivery system or tool.

In some other embodiments, the force of the nerve pressing against the biased arm 50 is sufficient to move the biased arm 50 and allow passage of the nerve. In various embodiments, the practitioner may stretch the nerve to reduce the diameter of the nerve, and the nerve may fit into an opening of the channel 102. The reduced diameter of the nerve allows the practitioner to slide the nerve into the channel 102, which opens slightly (i.e., to an open configuration) to allow for passage of the nerve. After the nerve passes through the channel 102, the medical practitioner stops the channel returns to its closed configuration and retains the unstretched nerve within the chamber 101. In the chamber 101, the medical practitioner stops stretching the nerve and it is thus retained in the chamber.

The process continues to step 1002, which couples the electrode to the somatic nerve (e.g., the perineal nerve) as discussed above. Accordingly, a medical practitioner may align the nerve with the channel 102 and position the perineal nerve through the channel 102. The nerve 200 may then be secured in the chamber 101.

In some embodiments, the nerve 200 may be stretched to have a thinner cross-sectional dimension to allow it to pass through the channel 102. The nerve 200, if briefly stretched, is released at the bottom of the chamber in the “free” area where the nerve is allowed to recover and return to approximately its original shape and size. However, in some embodiments, the gap of the channel 102 is large enough to allow the unstretched nerve 200 to pass. When coupled in the chamber 101, depending on its size, the nerve may relax or return toward its normal state.

The process then proceeds to steep 1003, which transitions the device to the closed configuration. As mentioned previously, the arm 50 may be biased closed. Thus, the medical practitioner need only remove the force used to open the arm 50, and the biasing force closes the channel 102 and moves the closed configuration. As mentioned previously, the closed configuration does not necessarily need to have a zero gap. In some embodiments, the gap is small enough to prevent the nerve from being dislodged from the chamber 101.

In various embodiments the movable arm 50 may be spring-loaded. Accordingly, the movable arm feature may also be referred to as a “clip,” that allows a nerve to rest in an uncompressed state in a chamber-like space, but with a gentle holding force pressing the nerve against electrodes. As the nerve moves down the channel, the spring force of the securing arm 50 returns towards the resting position and gently holds the nerve 200 in place. (Alternatively, the spring-loaded securing arm 50 could be held open until the nerve is fully seated in the chamber prior to returning to the resting position.)

The channel can accommodate a large variety in size of the nerves 200. The inventors have determined that the movable arm of various embodiments may effectively accommodate nerves 200 that vary in cross-sectional dimension by up to 400% or more (e.g., 1 mm nerve to 4 mm nerve). Various embodiments leverage the channel-chamber orientation where the channel as defined by the spring-loaded arm accommodates a stretched nerve 200 rather than being “held” in place by the clip, so the spring loading is not putting undue stress on the nerve. The nerve 200 travels down the channel (e.g., a variable width channel) depending on the position of the spring-loaded arm and rests in a chamber area that is also variable to the size of the nerve due to the same spring-loaded arm. As the channel is held gently closed by the spring force, the features of the chamber wall are oriented to help guide the nerve to maximize contact to the electrodes.

Thus, it should be understood that in the closed configuration, the nerve 200 may be slightly compressed by the arm 50 (e.g., by the nerve contacting surface 58). However, in some other embodiments, the nerve 200 may not be compressed by the arm 50, but merely trapped within the chamber 101.

As noted above, the nerve preferably is directly and conductively coupled with the electrode 104. Accordingly, there preferably is no other non-negligible organic component of the patient between the nerve and the device 100 (e.g., the electrode 104 and the nerve). Some embodiments may have a coating or other structure on the electrode 104 and still have a direct connection or coupling with the nerve.

The process then proceeds to step 1004. With the nerve securely in the chamber 101, the signal generator may begin transmitting the OAB control signal to the device 100, through the electrode 104, and to the perineal nerve (step 1004). Over time this signal reverses and normalizes the OAB symptoms. The stimulation activates the pelvic floor in a prescribed manner. During experimentation, however, the inventors were surprised to discover that this signal seemed to mitigate the effect of the natural OAB signal on the pelvic floor. Specifically, the patient's body naturally sends an OAB signal (referred to as the “natural OAB signal”), which is a part of the patient's mechanism that triggers symptoms of OAB. This activation on the perineal nerve, however, seems to block or otherwise interfere with the body's reaction to the natural OAB signal, blunting or otherwise mitigating the impact of the natural OAB signal.

As noted above, for a single session, the method may transmit the OAB control signal to the perineal nerve for between about 10 and 30 minutes. As noted, this timing can be fine-tuned as a function of the patient, severity of the disease, etc. After the OAB signal treatment is complete, then the process may continue to optional step 1006 to also treat SUI. Additionally, or alternatively, the process may send an FI signal to treat FI. Specifically, if the patient also suffers from SUI, then the method may transmit the SUI control signal to the perineal nerve, in this session, for between 30 and 120 seconds. Additionally, or alternatively, the process may send an FI signal to treat FI. Other embodiments may swap the order of steps 1004 and 1006.

The process then proceeds to step 1007, which removes the nerve from the chamber 101. Some embodiments may be implanted permanently, and may therefore skip this step. However, advantageously, various embodiments may be easily removed. As described previously, the device is transitioned to the open configuration. In some embodiments, a medical practitioner may do this using a tool. In some other embodiments, the nerve may be stretched by the medical practitioner and may pass through the channel 102. The force of the nerve may slightly open the channel 102 to the open configuration. After the nerve is removed from the chamber 101, the channel 102 may return to its biased closed configuration.

Although various embodiments refer to a movable arm that transitions between an open and closed configuration, some embodiments may have a non-movable arm or stationary arm. In such embodiments, the channel 102 may be configured such that the nerve may pass therethrough atraumatically when stretched. To that end, the material of the arm 50 (e.g., the buffer layer) may be sufficiently soft that it deforms to allow passage of the nerve 200.

Some or all of this process may be implemented in hardware, software, or both. For example, some or all of this process may be implemented using a custom application specific integrated circuit, FPGA, microcontroller, or other logic, with software (e.g., firmware), and integrated with the rest of the system.

Accordingly, the inventors discovered that they could treat both OAB and SUI via a relatively easy to access somatic nerve (e.g., the perineal nerve). Advantageously, this nerve is quite small, thus requiring a much lower power than required for many other conventional neurostimulation systems.

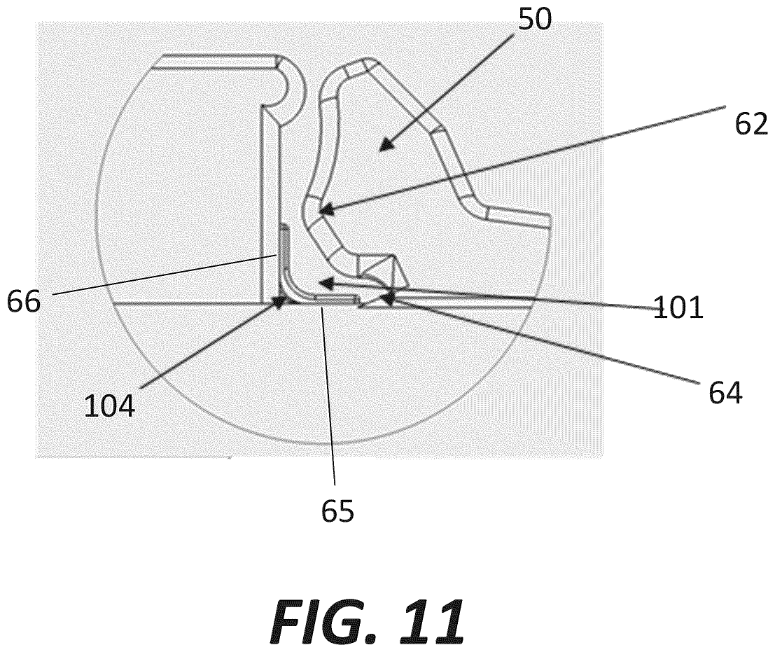

FIG. 11 schematically shows a close-up view of the arm 50 in accordance with illustrative embodiments of the invention. The device is shown in the closed configuration. As mentioned previously, the closed configuration is designed to gently hold the nerve 200 in the chamber where the two-sided electrode 194 is located. The benefit of this gentle holding force is to maintain direct contact with the nerve 200 either on the wall 66 or floor 65 of the chamber 101 where the electrode 104 is located and to reduce the channel 102 size for smaller nerve 200 sizes. In some embodiments, the wall 66 may have a total height of 1400 um from the floor to the top of the channel. The electrode 104 shape and size may be shaped as a curved “L” to promote direct contact regardless if the nerve 200 is at the larger or shorter end of the size range.

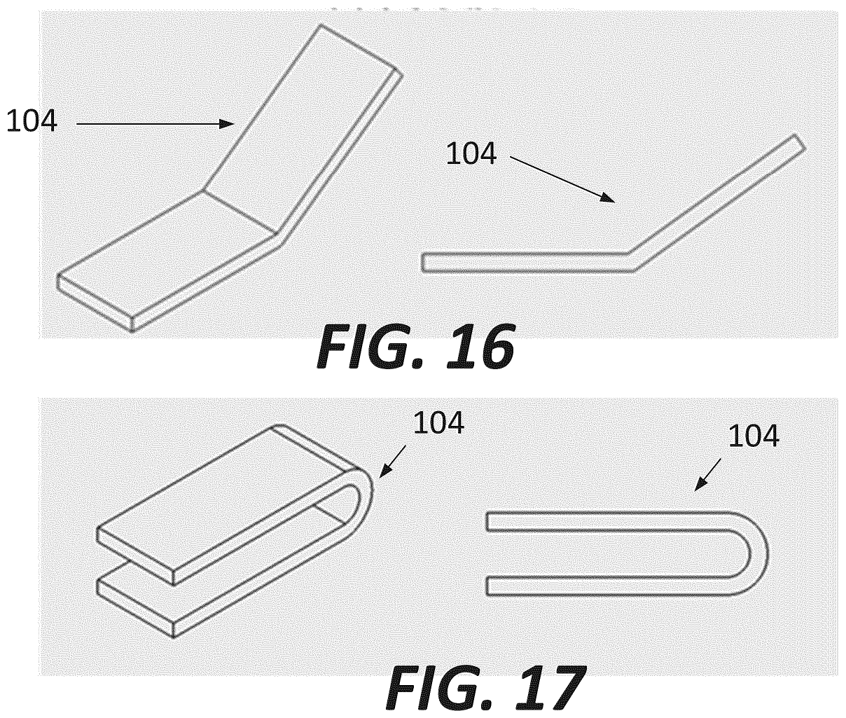

The main protrusion 62 in the spring-loaded securing arm 50 has two purposes: one, to provide a reduced channel 102 width to help secure the largest nerves 200 in the chamber 101 after traveling down the channel 102; and two, to provide gentle resistance to movement of the nerve 200 to hold it into the electrode 104. The angle of this protrusion 62 is angled to that it is gently encouraging the nerve 200 towards the electrode. In various embodiments, the protrusion 62 may have a 430 micron length. The protrusion 62 may be paired with a two sided or angled electrode 104 (e.g., see FIG. 16) in order to accommodate the spring-loaded securing arm's 50 change of angle as the size of the nerve varies. The smaller nerves 200 are encouraged downward into the horizontal portion of the electrode. The largest nerves 200 are encouraged more towards the vertical portion as this angle shifts for nerve sizes. A breakdown of these angles and distances are outlined below for nerve sizes having a diameter from 100 μm to 400 μm.

In various embodiments, the spring-loaded securing arm 50 may be primarily composed of an elastomeric material such as a silicone or plastic. Various embodiments may form at least a portion of the arm 50 (e.g., outer coating) from a Shore A durometer of 30-50 material. Additionally, while a soft nerve interface material is ideal, such as silicone, the spring-loaded arm 50 may have an overmolded or insert molded component embedded in the silicone to provide further spring force, especially in larger applications. Some options for this inserted component are nitinol springs, stainless springs, other elastomeric materials, or overmolded springs, and other similar materials in alternative durometers. Additionally, layers of various materials with different elastomeric materials can also provide the needed force. The material preferably is biocompatible for long term implants. Some embodiments may form the arm from silicone, polyurethane, embedded nitinol and/or stainless steel spring, etc.

Relief spaces 64 for nerve swelling may also be included on either side of the electrode 104. The nerve 200 is flexible and acts more as if it is a water balloon than a cable. If the nerve 200 experiences trauma during implantation, it swells and uses the relief space 64 to expand into as to not experience enough pressure to cause further damage. During swelling, or in the case there is temporary swelling beyond the ability of the clip to accommodate the change of nerve size, there is one or more relief spaces 64 for the nerve to expand into to ensure the nerve does not become further damaged. This relief space 64 provides an area of relief for any swelling or pressure build up without interfering with the electrode-nerve contact.

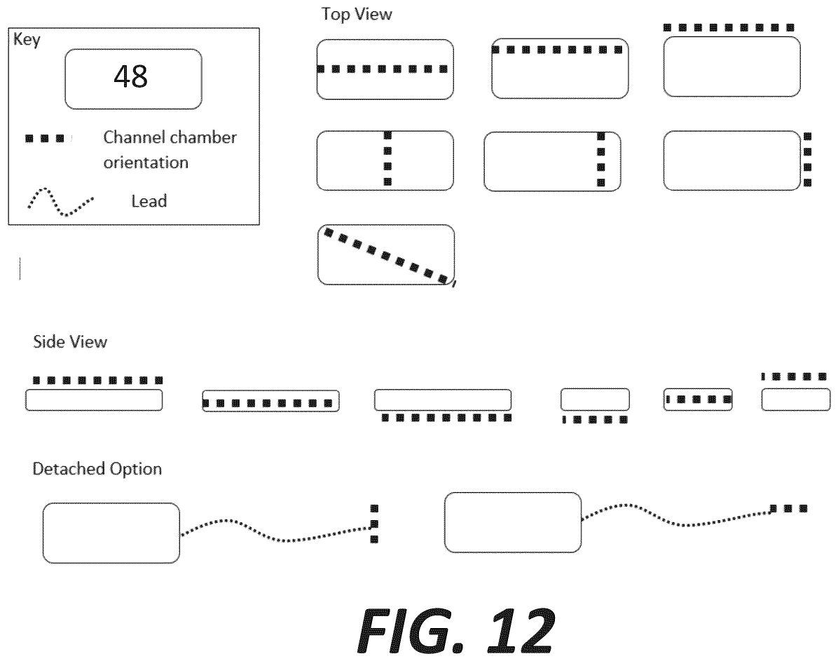

FIG. 12 schematically shows a plurality of orientations for the device body and the channel/chamber orientation in accordance with illustrative embodiments of the invention. The orientation of the channel 102 and the chamber 101 can be horizontal, vertical, or at any angle to the housing 48/body 40 of the neuromodulation device. Additionally, the channel 102 and the chamber 101 can be centered, or positioned to one end of the body 40 as desired to meet various anatomical requirements. Alternatively, some embodiments may separate the channel/chamber structure from the body 40 of the implant and utilize this at the end of a lead or lead extension to improve the implantation time of a traditional neurostimulator.

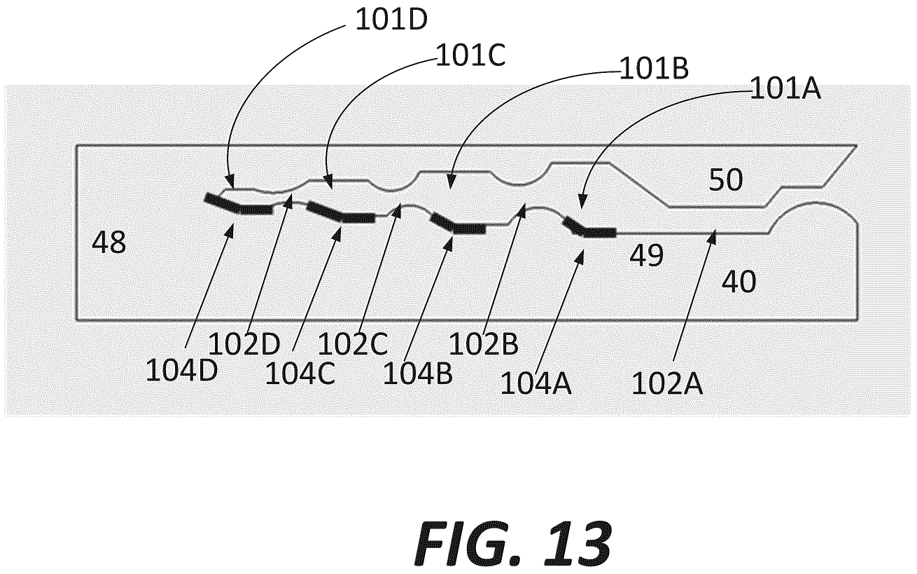

FIG. 13 schematically shows a device having multiple electrodes 104A-104D and chambers 101A-101D in accordance with illustrative embodiments. The various chambers 101A-101D may accommodate a variety of nerve 200 sizes FIG. 13 also shows a different orientation of the channel 102 relative to the main body 40 of device. Two sided electrodes 104A-104D may be positioned in each chamber 101A-101D.

Having a series of channels 102A-102D and chambers 101A-101D (e.g., that get progressively smaller) advantageously allow the stretched nerve 200 to enter into the appropriate chamber 101A-101D based on the nerve size. This arrangement accommodates a larger range of nerve sizes. The increasing bump outs provide the force to press the nerve 200 into the electrode located in the chamber, but also prevents or inhibits the larger nerve from passing through to the smaller chamber 101 and simultaneously prevents or inhibits the relaxed nerve from exiting the chamber it currently resides in. Additionally, or alternatively, some embodiments may include a flap or latch (e.g., at the entrance to the channel 102 or the entrance to the chamber 101) to further secure the nerve in the chamber. The flap (not shown) may formed of silicone to maintain the device in the closed formation. The latch or securing features may be a latch or securing feature added to the open end.

The device 100 and signal generator may be distributed separately, or together as a kit.

It should be apparent that various embodiments provide a number of advantages. For example, electronics that are otherwise susceptible to fluid ingress and that are non-biocompatible are reliably separated from the aqueous in-vivo implantation environment. Furthermore, the package 46 and/or housing 48 are formed from materials that are selected for transmission of RF energy. The silicone overmolded housing 48 allows for complex shapes to be manufactured. The silicone may be processed at higher temperature than the electronic components may otherwise endure (e.g., if they were to be injection molded). In various embodiments, the device 100 has a reliable hermetic enclosure, while simultaneously having a desirable (e.g., complex) outer geometry and durometer to engage with the tissue inside the body (e.g., the nerve 200).

As additional examples, illustrative embodiments are easy to implant, i.e., the nerve slides into the chamber with minimum force. Illustrative embodiments also reduce surgery time and avoid trauma, and may be implanted in a smaller space and with minimal alteration to the tissue relative to a typical electrode cuff. Furthermore illustrative embodiments may be suture-free (i.e., does not need complicated suturing to secure nerve—easily deployed). Furthermore, direct contact with the nerve is promoted by the orientation and direction of the gentle force of the arm 50, which results in the electrical signal directly adjacent to the nerve reducing power consumption, maximizing stimulation, and reducing tissue ingrowth between nerve and electrode reducing the degradation of the signal.

In general, pressure for positioning the largest sized nerves in the chamber is well below the generally accepted acute limit of 30 mmHg for the largest nerves within the design window. In some embodiments, the nerves may have a cross-sectional dimension (e.g., diameter) of between about 0.5 mm and about 3.0 mm. However, various embodiments may receive larger nerves or even smaller nerves. Pressure for holding force is also well below generally accepted chronic limit of 20 mmHg for the largest nerves within the design window. To confirm this, the inventors performed a variety of tests. The pressure of the device on the nerve was performed on a 10× scaled model. A pressure sensor was combined with a fluid filled phantom nerve. The nerve was inserted and removed from the device and maximum and holding pressures were both recorded (the insertion of the nerve was worst case and the clip was not manually opened, but the nerve felt the full force of the clip during insertion). Maximum force recorded was 15 mmHg and maximum holding force recorded was 3.1 mmHg

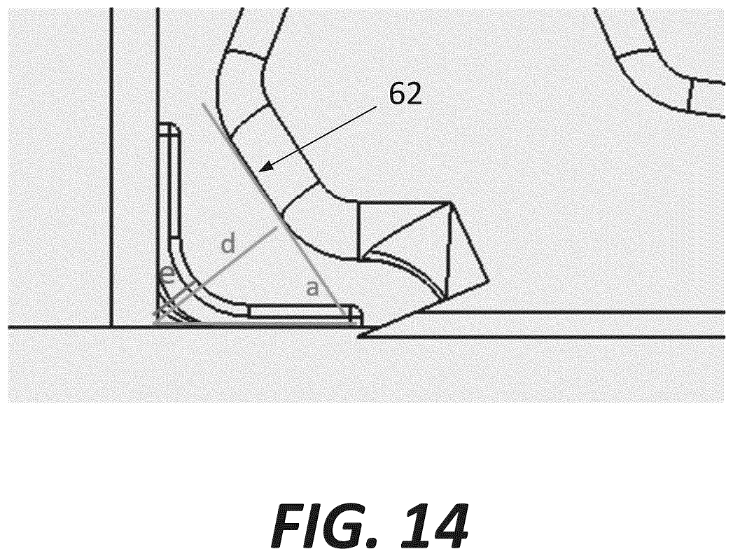

FIG. 14 schematically shows details of the protrusion 62 on the arm 50 in accordance with illustrative embodiments. As the size of the nerve changes from 100 μm to 400 μm, the angle of the protrusion 62 on the spring-loaded securing arm may change to accommodate the large discrepancy in size. The calculations for the example of 100 μm to 400 μm are shown below and can be scaled for alternative nerve ranges. The example provided below assumes the following:
the channel in the open configuration is 300 μm wide when fully opened,
the part of the bump feature in the channel is slightly larger than the largest configuration size 430 μm,
the electrode is 450 μm in the vertical and horizontal directions,
and the overall height of the channel and chamber is 1.4 mm.

Electrode distance from corner (e): 0.13 mm

| | | | | Distance between |
|---|---|---|---|---|
| Nerve size - | Angle of | | | corner of electrode |
| Diameter at | bump out | | Distance from | and bump out on |
| rest (μm) | (a) | | corner (d) | arm (d − e) |
| 100 | 47.8 | degrees | 0.23 mm | 0.1 mm |
| 200 | 50 | degrees | 0.33 mm | 0.2 mm |
| 300 | 57 | degrees | 0.43 mm | 0.3 mm |
| 400 | 73 | degrees | 0.53 mm | 0.4 mm |

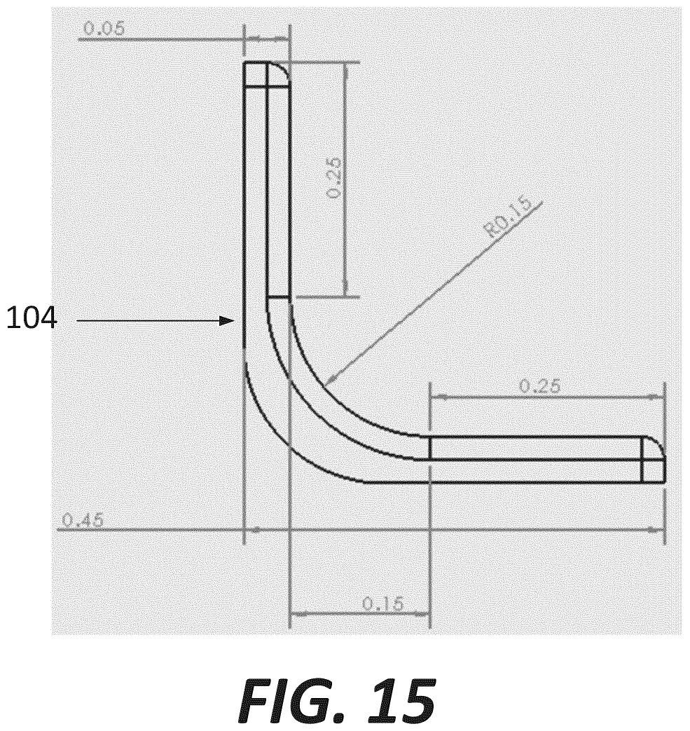

FIGS. 15-17 schematically show various details of the electrode 104 in accordance with illustrative embodiments of the invention. The electrode 104 as discussed may have both a horizontal and vertical surface to accommodate the various angular engagement of the pressing portion of the arm 50 to the nerve. The surface area of the electrode 104 preferably accommodates the needed charge density on each face of the electrode 104. FIG. 15 schematically shows a curved electrodes in accordance with illustrative embodiments. It should be apparent that the above mentioned dimensions are merely exemplary.

FIG. 15 schematically shows an angled electrode in accordance with illustrative dimensions. The angle between the two portions of the angles electrode 104 may be adjusted. Curved or folded electrodes may provide better direct contact with the nerve than a traditional one dimensional electrode. This allows flexibility as the angle of the pressure on the nerve changes depending on the size of the nerve so that either a horizontal force and vertical force both result in intimate contact with the conductive surfaces. Alternatively in a different orientation the two-sided electrode can be configured not only in a “L” shape but at various internal angles to accommodate different electrode orientations. FIG. 17 schematically shows a folder electrode 104 in accordance with illustrative embodiments. The folded electrode 104 configuration has two sides of the electrode running both sides of the nerve to “sandwich” the nerve in between two sides of the electrode.

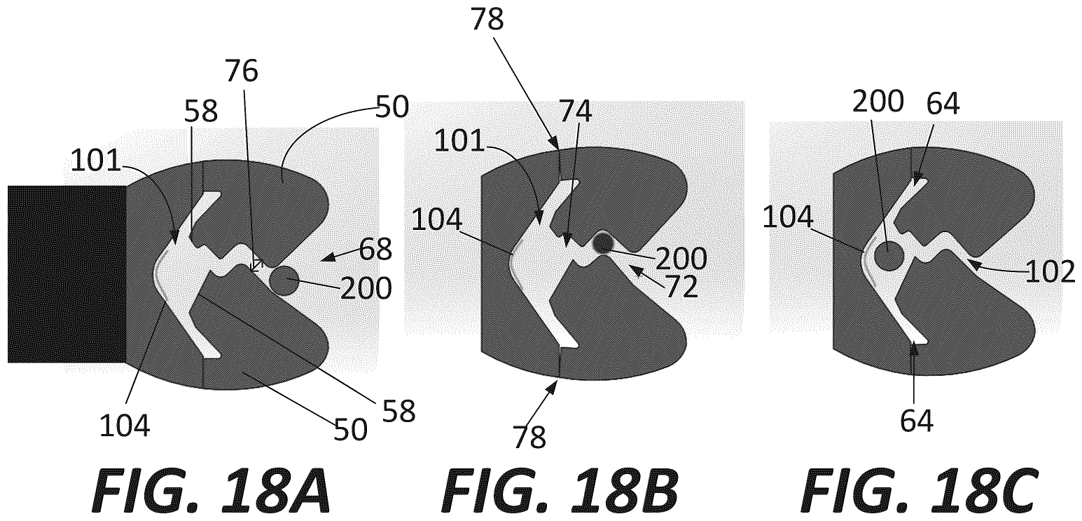

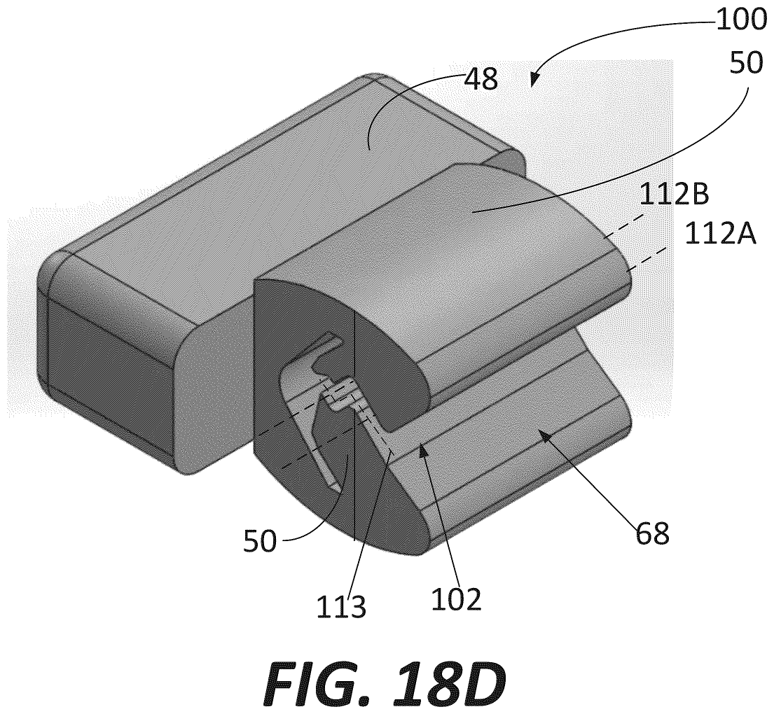

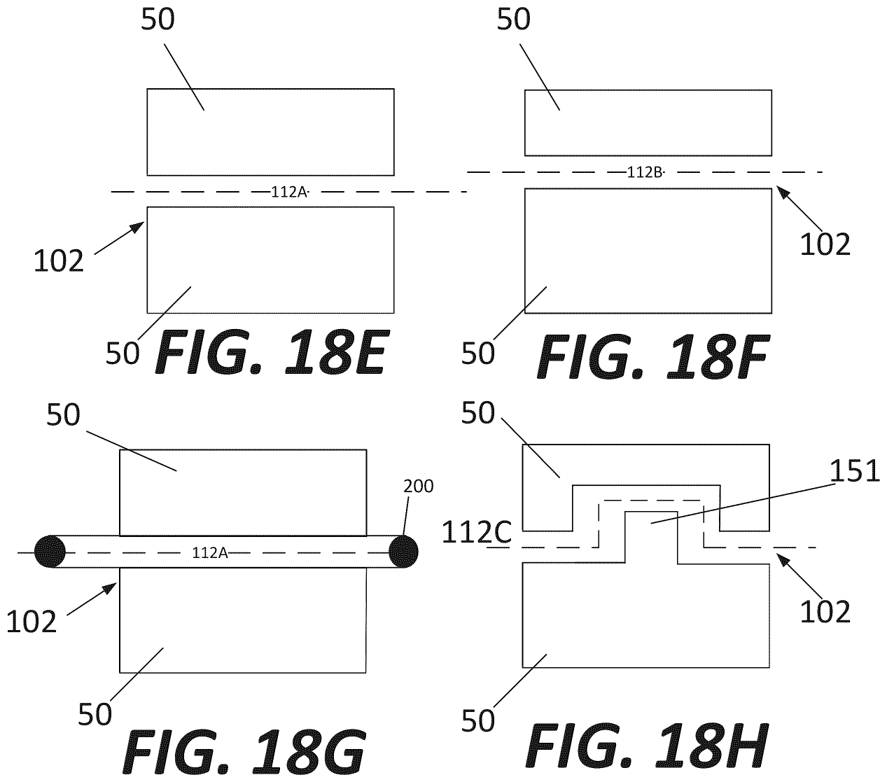

FIGS. 18A-20C schematically show an alternative embodiment of the neuromodulation device 100 in accordance with illustrative embodiments. Specifically, FIGS. 18A-20C show an embodiment having a plurality of jaws 50 (e.g., formed from silicone or including a silicone overmold as discussed previously). The main body 40 is omitted in some of these figures, but it should be understood that various embodiments may include the main body 40, the housing 48 (e.g., ceramic), and/or other components described previously. However, contrary to previously shown embodiments having the channel 102 opening oriented substantially perpendicular to the main body 40, FIGS. 18A-20C show the channel 102 opening oriented substantially parallel to the longitudinal axis of the main body 40. However, in other embodiments, the channel 102 may be oriented in any direction (e.g., diagonal to the main body 48). It should also be understood that the orientation relative to the main body may depend on the orientation and dimensions of the main body.

In some embodiments, the jaws are directly coupled to the main body 40. Thus, the chamber is at least in part formed from the main body 40 (e.g., by the package 46 and/or the housing surrounding the package 46). Alternatively, the jaws 50 may be separately coupled to the main body by an attachment portion. For convenience, the main body 40 and the housing 48 are not shown in FIGS. 18A-20C, but it should be understood that various embodiments of the jaws 50 may be coupled to the housing 48 and/or package 46.

Advantageously, a single device 100 is configured to couple with and stimulate nerves 200 of a variety of sizes (e.g., between 0.5 mm and about 4 mm).

Although the main body 48 is shown in FIG. 18A as being smaller than the jaws 50, it should be understood that the drawing is not necessarily to scale. In some embodiments, the main body 48 may be larger or smaller than the jaws 50. Additionally, in various embodiments, the main body 48 may form part of the chamber 101 having the electrode 104. In some other embodiments, however, the main body 48 may not form part of the chamber 101. Instead, the jaws 50 and/or an intermediary between the jaws 50 may define the chamber 101.

FIGS. 18A-18C schematically show a process of positioning a small nerve 200 (e.g., about 0.5 mm to about 1.5 mm in diameter) within the chamber 101 for stimulation. The figures show a side view of the device 100.

For the sake of discussion, the nerve shown in FIGS. 18A-18C is a small nerve of about 1 mm in diameter. Together, the jaws 50 define a V-shape funnel 68 on the exterior of the device 100 that is configured to assist a surgeon with positioning the nerve 200 at the proximal end 72 of the channel 102. When the nerve 200 is positioned at the proximal end 72 of the channel 102, the nerve 200 preferably has a greater cross-sectional dimension than the channel 102. For example, the channel 102 may have a cross-sectional dimension of between about 0.45 mm and about 0.75 mm. However, in some embodiments, the channel 102 may be completely closed (e.g., a dimension of about 0 mm). Advantageously, the reduced channel dimension 76 prevents accidental dislodgement of the nerve 200. Additionally, to that end, the channel 102 may have non-linear or tortuous shape to assist with preventing accidental dislodgement of the nerve 200. However, some embodiments may include a linear channel 102.

In any event, as shown in FIG. 18B, the nerve 200 may be stretched (e.g., by the surgeon applying a force) and/or squeezed (e.g., by the inner surface of the channel 102) to reduce the cross-sectional dimension of the nerve 200. Preferably, the reduction in nerve 200 dimension is less than 50% to prevent or reduce damage to the nerve 200 that might otherwise occur from greater reductions in cross-sectional size. For example, a 1 mm nerve 200 may be reduced to a cross-sectional dimension of about 0.75 mm. Assuming the channel dimension 76 is about 0.5 mm, the nerve 200 may still be too large to pass through the channel 102. To that end, the device 100 may include hinges 78 that allow the jaws 50 to open and thereby expand the channel dimension 76. In some embodiments, the hinges 78 are formed by a deformable material (e.g., silicone) that allow the jaws 50 to open outwardly. Additionally, or alternatively, the channel 102 may include a deformable and/or resilient wall or coating that provide for expanding the channel dimension 76.

As shown in FIG. 18C, when the nerve 200 passes the distal end 74 of the channel 102 and enters the chamber 101, the jaws 50 may again close. Because of the reduced channel dimension 76 and the non-linear channel 102 shape, the small nerve 200 is securely coupled within the chamber 101. Although not drawn to scale, the jaws 50 may include nerve contact surfaces 58 configured to press and/or hold the nerve 200 against the electrode 104. In various embodiments, the jaws 50 may be biased to return to a close position, and/or the deformable material may be resilient and return to its original dimensions.

FIG. 18D schematically shows a perspective view of the device 100 (with details of the chamber 101, such as the electrode 104 omitted). The channel 112 has a particular travel path 113 (also referred to as a central axis 113) for the nerve 200. For example, as shown in FIG. 18D, the central axis 113 is non-linear. The central axis 113 may be defined by the arms 50.

In addition to the central axis 113, the device 100 has a longitudinal axis 112 that is orthogonal to the central axis 113 at any given point. During nerve implantation procedure, the nerve 200 is generally parallel to the longitudinal axis 112 as it travels along the central axis 113. Two different longitudinal axes 112A and 112B are shown for two different points along the central axis.

FIG. 18E schematically shows a cross-sectional view of the longitudinal axis 112A of FIG. 18D. As shown the channel 102 is axial, such that the longitudinal axis 112A is uninterrupted. This allows for the nerve 200 to pass through the channel 102 with minimal manipulation by the surgeon. In some embodiments, the nerve 200 may be stretched to pass through the channel 102 and/or the channel walls formed by the jaws 50 may be deformable. FIG. 18G schematically shows a nerve passing through the channel 102 at the cross-section of axis 112A.

FIG. 18F schematically shows a cross-sectional view of the longitudinal axis 112B of FIG. 18D. FIG. 18F shows a different cross-section from the view of FIG. 18D. However, again, the channel 102 is axial, such that the longitudinal axis 112B is uninterrupted. The channel may thus be said to be continually axial, i.e., the longitudinal axis 112 at any given point along the central axis 113 is uninterrupted.

FIG. 18H schematically shows an alternative embodiment from FIGS. 18E-18G. In particular, the longitudinal axis 112C is interrupted (e.g., by a tooth 151). In order for the nerve 200 to pass through the channel 102 of FIG. 18H, the surgeon must perform a very careful manipulation of the nerve 200 (e.g., to conform to the shape of the axis 112C), force the nerve through by application of traumatic pressure, or actively open the jaws 50 (e.g., with a separate tool). Although FIG. 18H does not have an axial longitudinal axis 112C at rest (as shown in the figure), when the jaws 50 are opened, the longitudinal axis 112C may become axial. However, illustrative embodiments advantageously have an axial longitudinal axis 112A, 112B, at rest, such that the nerve may easily be slid into the chamber 101.

Accordingly, some embodiments have a non-linear channel 102 (i.e., along central axis 113) that is continually axial, as shown in FIGS. 18D-18F. Advantageously, the nerve may pass through the channel 102 with minimal manipulation, atraumatic force, and without requiring a separate tool to open the arms 50.

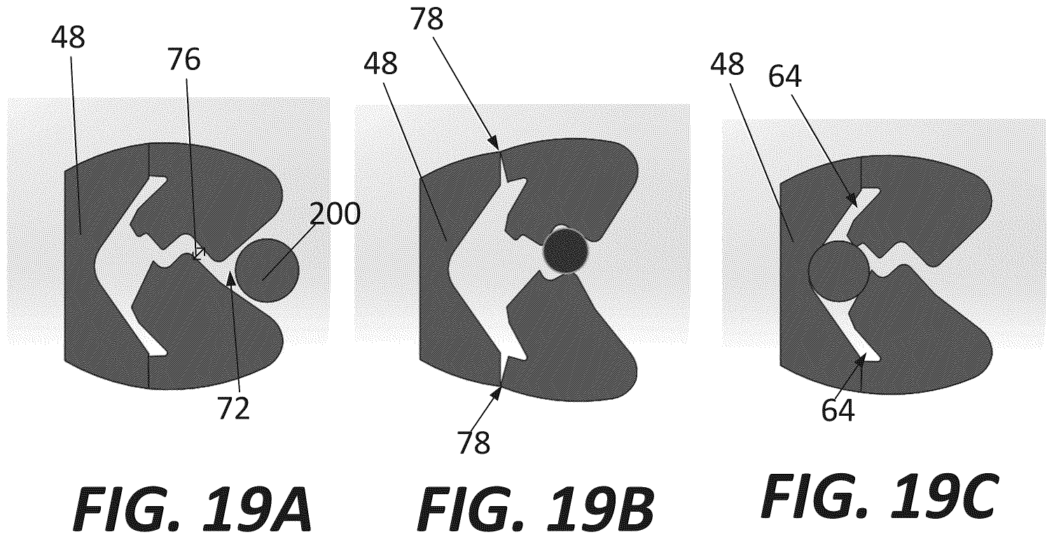

FIGS. 19A-19C schematically show a process of positioning a medium nerve 200 (e.g., about 1.5 mm to about 2.5 mm in diameter) within the chamber 101 for stimulation. The figures show a side view of the device 100.

For the sake of discussion, the nerve 200 shown in FIGS. 19A-19C is a medium nerve of about 2 mm in diameter. Similar to the process described previously, the V-shape funnel assists with positioning the nerve 200 relative to the proximal end 72 of the channel 102. For the sake of discussion, the nerve 200 may be stretched to reduce the nerve diameter to about 1.5 mm. As described previously, the channel dimension 72 may be about 0.5 mm. Accordingly, the nerve is considerably thicker than the channel 102 is wide when the jaws 50 are in the resting position. The jaws 50 may be expanded open to increase the channel dimension 76 and to allow the nerve 200 to pass through the channel. To that end, illustrative embodiments may include a delivery device with cam features that press the jaws 50 open via deformation and/or hinging. Although FIG. 18B schematically shows the hinge splitting open, various embodiments do not have a hinge opening (which might otherwise trap a portion of a larger deformed nerve).

As shown in FIG. 19C, the nerve 200 comes to rest in the chamber 101 and the jaws 50 may be reclosed. For example, the nerve 200 relaxes and returns to its 2 mm diameter. Additionally, the silicone of the jaws 50 may relax and the channel dimension 76 is restored to about 0.5 mm, preventing the nerve from backing out of the channel 102. In some embodiments, the jaws 50 are biased to a resting position (e.g., where the channel 102 has a dimension 76 of about 0.5 mm or to a fully closed position). Additionally, or alternatively, the delivery device may close the jaws 50. The silicone deformation and the small Z channel 102 securely couple the medium sized nerve in the chamber 101.

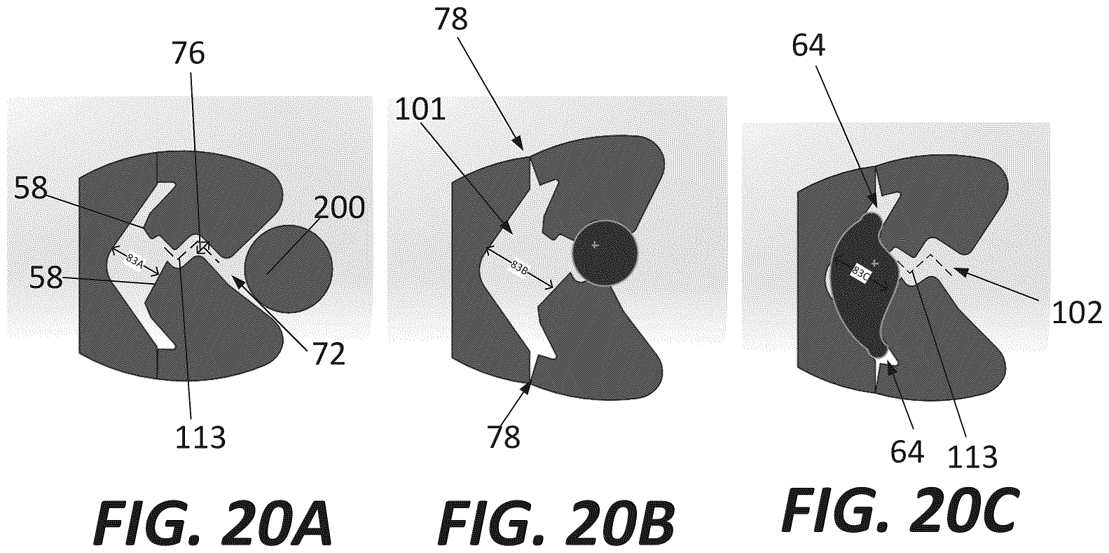

FIGS. 20A-20C schematically show a process of positioning a large nerve 200 (e.g., about 2.5 mm to about 3.5 mm in diameter) within the chamber 101 for stimulation. The figures show a side view of the device 100. As shown in FIG. 20A, the device 100 is considered to be at rest or in a resting position. In the resting position, the device is at steady state, i.e., no forces are being applied to the device to open the jaws 50 (e.g., either by a nerve or by a tool). Thus, in various embodiments, the resting position is the “closed position” of the device. As mentioned previously, the closed position does not imply that the channel or chamber need to be closed. Indeed, as shown, in various embodiments the device has an open channel 102 at rest. Furthermore, the channel 112 may be continuously axial when the device is at rest (and when the device is transitioning to its open configuration). Some embodiments may close the channel (e.g., using a flexible flap or valve with a very low cracking pressure). Such closure may be applied along the channel 102 without limiting the ability to use the slide-and-lock nerve position method.

For the sake of discussion, the nerve 200 shown in FIGS. 20A-20C is described as a large nerve of about 3 mm in diameter when undeformed. The nerve 200 may be positioned at the proximal end 72 of the channel as described previously. The device 100 may have a chamber dimension 83A (e.g., such as a maximum chamber dimension or a minimum chamber dimension) without the nerve 200 therein.

As shown in FIG. 20B the nerve 200 may travel along a central axis 113 of the channel. In some embodiments, the nerve 200 is stretched (e.g., by the surgeon) and/or squeezed (e.g., by the inner surface of the channel 102) to reduce the cross-sectional dimension of the nerve 200. Preferably, the reduction in nerve 200 dimension is less than 50% to assure safety to the nerve 200, as some damage might otherwise occur from greater reductions in cross-sectional size. For example, the nerve may be reduced to a cross-sectional dimension of about 2.25 mm. Assuming the channel dimension 76 is about 0.5 mm, the nerve 200 may still be too large to pass through the channel 102. To that end, the hinges 78 open the jaws 50 and expands the channel 102 dimension 76 to allow the large nerve 200 to pass through the channel 102. Accordingly, various embodiments have an adjustable size and/or shaped chamber 101. Similarly, the chamber dimension 83B may be adjusted by movement of one or more arms 50.

As shown in FIG. 20C, when the nerve 200 passes the distal end 74 of the channel 102 and enters the chamber 101, the jaws 50 may again close. However, because of the large size of the nerve 200, the jaws 50 may or may not be able to close entirely so as to return the channel 102 to its original dimension 77. In a similar manner, the arms 50 may or may not be able to close entirely to return the chamber 101 to its original dimensions 83A. The chamber 101 may have a new adjusted chamber dimensions 83C when the nerve 200 is positioned in the chamber 101. Regardless, the jaws 50 retain the large nerve 200 within the chamber 101. To that end, the jaws 50 may include relief spaces 64 (also referred to as deformation areas 64) where the nerve 200 shape may deform and expand when compressed (e.g., such that the cross-sectional area of the nerve is not reduced as much as if there were no deformation areas 64). In various embodiments, the nerve 200 may deform and/or expand in the one or more relief spaces 64 so as to reduce the pressure applied to the nerve. The device may configured such that the less than 4 kPa of pressure is applied to the nerve, accounting for the nerve relief space 64. Additionally, to assist with reducing pressure applied to the nerve 200, the contact surfaces 58 may be configured to deform, thereby giving the nerve 200 additional room to expand within the chamber 101. Furthermore, in some embodiments, and as shown in FIG. 20C, the channel 102 dimension 76 may be expanded in the resting configuration because of interference with the large nerve 200.

Because of the reduced channel dimension 76 and the non-linear channel 102 shape, the small nerve 200 is securely coupled within the chamber 101. Although not drawn to scale, the jaws 50 may include nerve contact surfaces 58 configured to press and/or hold the nerve 200 against the electrode 104.

It should be understood that various embodiments advantageously allow the nerve 200 to expand/deform within the chamber 101, as opposed to a compensatory expansion/deformation that might otherwise occur outside of the chamber 101 and damage the nerve 200. However, in various embodiments, some deformation outside of the chamber 101 may occur. Preferred embodiments advantageously provide jaws with deformable nerve contact portions, as well as designated relief spaces 64 within the device to allow for deformation adjacent/local to the area of compression on the nerve. As is known in the art, nerves are made up of bundles of axons. By allowing localized deformation of the nerve, the position of individual axons and/or fascicles may be rearranged without necessarily damaging any individual axon (as might occur if the nerve were to drastically compress or stretch.

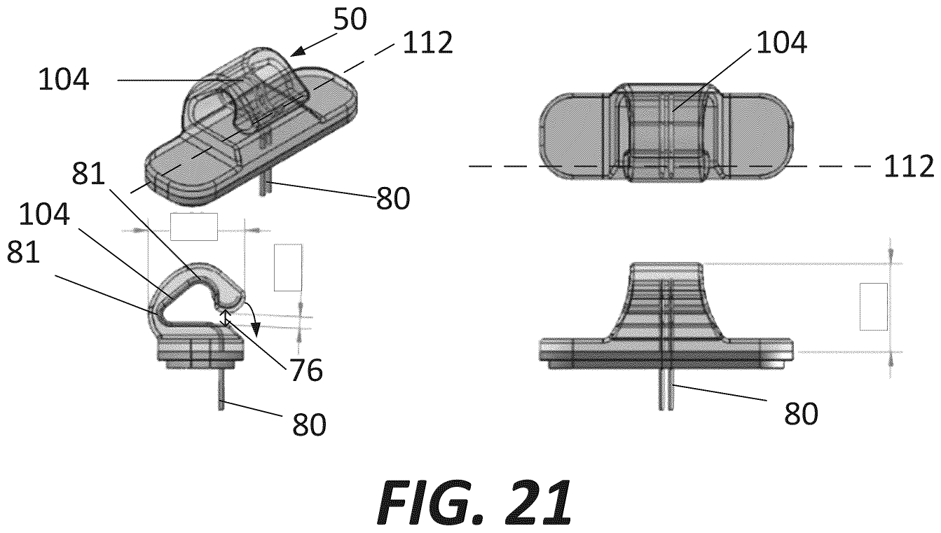

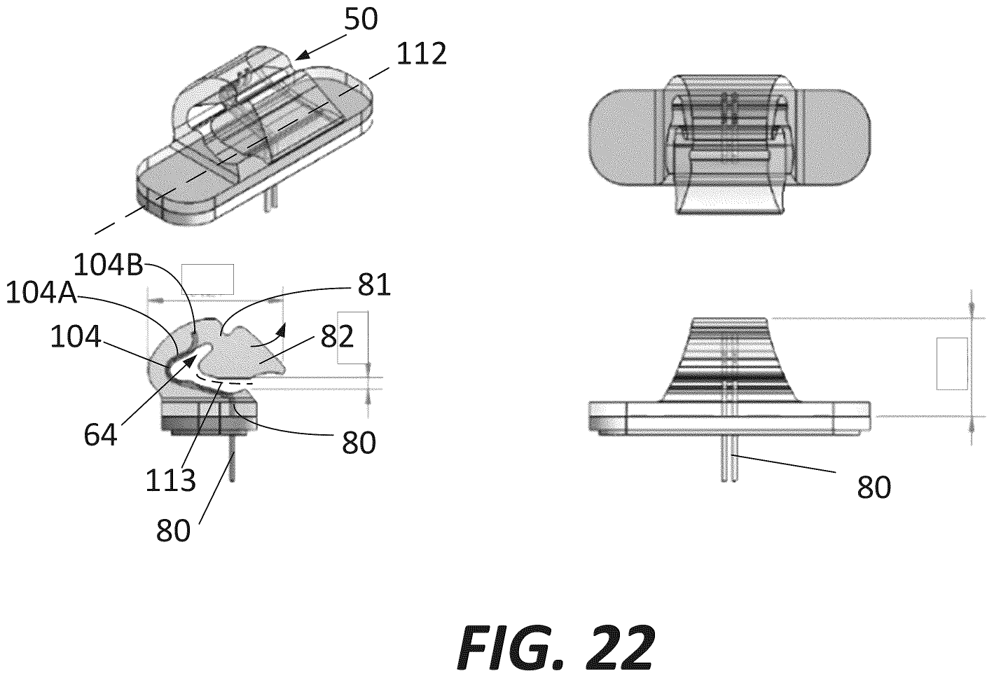

FIGS. 21 and 22 schematically show the neuromodulation device 100 in accordance with illustrative embodiments. FIG. 21 shows four different views of a neuromodulate device 100. The hermetically sealed main body 40 is largely omitted. However, a feedthrough conductor 80 that extends from the interior of the hermetically sealed main body 40 is shown. The feedthrough conductor 80 extends through the package 46 and the buffer layer 49. The feedthrough conductor 80 then forms the electrode 104 within the chamber 101, which is defined by the movable arm 50.

The movable arm 50 is shown biased towards a closed position. Although referred to as the “closed position,” some embodiments may have the gap 76. Therefore, it is not necessary that the movable arm 50 entirely close the gap 76 in the closed position. The closed position 76 is used to refer to the position of the channel 102 when the channel is narrow to retain the nerve. As the nerve passes through the channel 102, the gap 76 increases (e.g., because the arm 50 moves and/or deforms) and the movable arm 50 transitions towards the open position. To help facilitate the opening of the channel, the movable arm 50 may include one or more bending points 81 formed from a material configured to bend, in order to accommodate the nerve 200. It should be understood that the gap 76 does not have to be fully closed to be biased towards the closed position. In a similar manner, the gap 76 does not have to be fully open to transition towards the open position.

As described previously (e.g., with reference to FIGS. 18A-18G), the device 100 defines an axial channel along a length of the channel 102. Advantageously, the continuously axial channel 112 that allows for ease of insertion and/or removal of the nerve 200, particularly using the slide-and-lock method described in U.S. patent application Ser. No. 16/414,169, which is incorporated herein by reference in its entirety. Some embodiments may use a non-axial channel 112, such that the longitudinal axis of the channel 112 changes directions along its length (e.g., FIG. 18H having interlocking teeth). However, embodiments having the axial channel 112 advantageously may use the slide-and-lock method, e.g., by stretching the nerve 200 axially and sliding it through the continuously axial channel 102. This method advantageously does not require the use of external tools or other forces (other than the pressure of the nerve pressing against the jaw 50) to help open the jaw 50 for the nerve to traverse the channel 102.

FIG. 22 shows four different views of an alternative to the movable arm 50 in accordance with illustrative embodiments. FIG. 22 shows a bending feature 81 that is configured to move for bigger nerves 200 as they pass through the channel 102 and/or as they settle in the chamber 101. However, the bending feature 81 (e.g., the neck) assists with keeping smaller nerves 200 pressed against the electrode 104. Advantageously, the neck 81 allows for a head 82 to pivot to accommodate various size nerves 200 in the channel 102 and/or in the chamber 101.

In some embodiments, the feedthrough conductor 80 gets welded to a separate electrode 104 at a welding point outside of the hermetically sealed package 46. However, various embodiments provide an unjointed (e.g., unwelded) electrode 104 that is formed by the feedthrough conductor 80. The tolerances for welding the electrode 104 onto a feedthrough pin 80 at this scale are difficult. By using an unjointed electrode 104, manufacturing issues are simplified. Furthermore, as shown, portions of the electrode 104 may be embedded within the arm 50. Thus, the electrode 104 may have an exposed electrode portion 104A and an embedded electrode portion 104B. For example, the embedded portion 104B may include the end of the electrode. In various embodiments, the exposed electrode portion 104A is configured to contact at least 20%, at least 25%, or at least 35% of the perimeter of the nerve 200 positioned within the chamber 101. In some embodiments, the exposed electrode 104A is configured to contact at least 40% of the perimeter of the nerve 200 positioned within the chamber 101.

As shown, various embodiments combine the nerve coupling portion (i.e., the chamber 101) and implantable pulse generator (e.g., within the package 46), advantageously providing a smaller device 100 and a simpler implantation procedure. Additionally, various embodiments do not require external leads between the nerve coupling portion and the IPG. Advantageously, a single unjointed feedthrough conductor 81 may extend from the hermetically sealed housing 46 into the chamber 100 to form the electrode 104. This is in contrast to devices where the feedthrough conductor 81 is welded to the electrode 104. Illustrative embodiments may use the feedthrough conductor directly as the electrode 104, thereby providing an unjointed or continuous electrode.

Prior art devices implant the IPG by exposing the nerve to place the cuff, then tunneling the lead using customized tools to the location of the IPG where a pocket is formed under the skin to place the IPG, The IPG is then connected to the leads and placed in the pocket. In contrast, illustrative embodiments provide a simpler procedure since the sizing is automatic to the nerve (within an acceptable range of approximately +/−50% to 75%, for a 2 mm nerve that is a range from 0.5-3.5 mm). Advantageously, there is no tunneling and no need to have a separate surgical location for the IPG.

Furthermore, the entire lead body is not needed along with the connector blocks, proximal connectors and other similar components. The feedthrough conductors 81 (e.g., extending from the hermetically sealed package 46) can be used as the electrode 104. The feedthrough conductors 81 may be formed, for example, from Pt—Ir material. Additional benefit of using this structure as the electrode 104 is that the feedthrough pin 81 can be bent and utilized to support the structure and holding force of the movable arm 50 to atraumatically couple the nerve 200.

Accordingly, illustrative embodiments provide a neuromodulation device where the feedthrough conductors 81 operates as both the electrodes 104 and structural components that are merged to achieve the appropriate flexion properties for the arm 50. The material properties are select so that nerve 200 is kept in close, but not traumatic, contact with the electrodes 104.

The movable and/or flexible arm 50 may adjust both during the insertion of the nerve in the channel 102 and when the nerve is positioned in the chamber 101. The arm 50 is configured to adjust the dimensions of the chamber 101 to capture smaller nerves (e.g., 0.5 mm, 1 mm) without putting traumatic pressure on the larger nerves 200 (e.g., 3 mm, 4 mm).

Illustrative embodiments are configured to retain nerves having a size of 0.5 mm to 4 mm in the chamber 101 with pressures of less than 6.7 kPa. Nerves 200 are relatively susceptible to damage from high pressures. Accordingly, illustrative embodiments form the arm 50 from materials that bias the arm to reduce pressure on the nerve 200 as it passes through the channel 102, and as it rests in the chamber 101. In particular, the arm 50 is configured to impart less than 6.7 kPa on the nerve 200 to prevent structural damage to the nerve. For example, the channel 102 preferably has a cracking pressure of less than 6.7 kPa. In various embodiments, the device 100 is configured so that less than 10 kPa or less than about 1.5 psi of pressure is applied to the nerve. Of course, some embodiments may provide higher pressures than disclosed herein. Depending on the amount of time the nerve 200 is in the channel 102, this pressure may be reduced. For example, if passing the nerve through the channel 102 takes 2 minutes or longer, the device 100 should be configured so that the less than 6.7 kPa of pressure is applied to the nerve. In a similar manner, the nerve 200 is situated in the chamber for long periods of time, and therefore, the device may be configured so that less than 4 kPa of pressure is applied to the nerve at any given time in the chamber. The device 100 may be configured to result in these pressures using, among other things, specific materials, relief spaces 64, and/or a plurality of arms 50.

Furthermore, illustrative embodiments are configured to retain nerves having a size of 0.5 mm to 4 mm in the chamber 101 with pressures of less than 4 kPa (or 30 mm Hg) to reduce undesirable long-term effects such as blocking of nerve function and/or edema. Thus, the chamber 101 preferably has a retention pressure of less than 4 kPa on nerves 200 that are between 1 mm and 3 mm in diameter (nerve diameter being a generally accepted size measurement of the nerve when the nerve is at rest and undeformed).

Bench testing of the device 100 has shown that the maximum insertion force for large nerves (e.g., 4 mm diameter) is less than 2 kPa (15 mmHg). and after the model nerve was resting in the chamber, the compression was on average 0.147 kPa (1.1 mm Hg), indicating the flexible channel/chamber approach is a viable approach.

Additionally, some electrodes implanted into nerves in clinical studies chronically compress them (approximately 40%). These are called “FLAT” electrodes (primarily the FINE electrode) and it has been reported an estimated intrafascicular pressure of less than 30 mm Hg can reshape the nerve without significant changes in the nerve physiology or histology. Flattening the nerves above 60% applies 60 mm Hg of pressure and can cause nerve damage. Early bench testing of the NeuroClip has shown, as mentioned in the previous paragraph, that chronic compression values much lower than the 60 mm Hg shown with the “flat” electrodes can be achieved.

Illustrative embodiments provide a neuromodulation device that is configured to atraumatically couple to a variety of nerve sizes, from 0.5 mm to 4 mm. As described herein, atraumatic coupling provides less than a 4 kPa sustained pressure on the nerve when the nerve is in the chamber (e.g., in the chamber for many hours, days, etc.). Furthermore, atraumatic coupling provides less than 6.7 kPa when passing through the channel 102 (e.g., pass through the channel for a short time period of less than 1 minute).

The device 100 is configured to accommodate a variety of nerve sizes (e.g., from 1 mm to 3 mm). The same approach can be scaled to multiple nerve size ranges.

To accommodate various nerve sizes, the device is sufficiently flexible enough to allow the largest nerves to pass through the channel portion and rest in the chamber, but not too large to allow the smallest nerves to easily slip out while maintaining contact with the electrodes. To that end, various embodiments use Nusil (NUSIL product with Class VI biocompatibility approval) silicone, a biomaterial that is widely used with long-term active implantable devices. This material flexes to accommodate the largest nerves passing through the channel 102, and also when the nerve is seated in the chamber to reshape into additional space provided to prevent a buildup of pressure inside the nerve. Instead of a large round nerve being overcompressed in the chamber, the nerve shape changes to fill the open space provided. The nerve 200 can endure moderate reshaping of cross-sectional shape without causing damage (e.g., 30% reduction in maximum cross-sectional area).

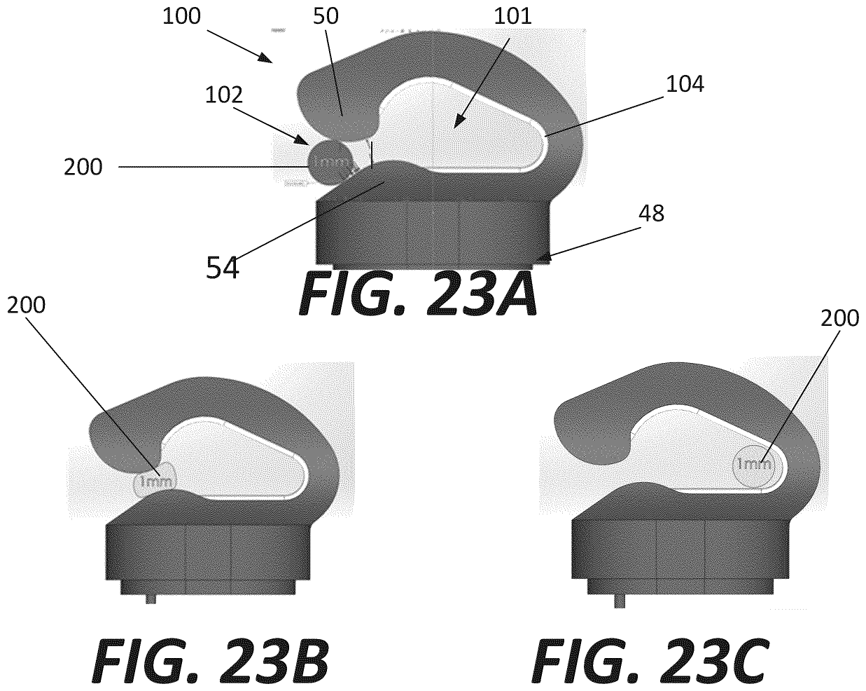

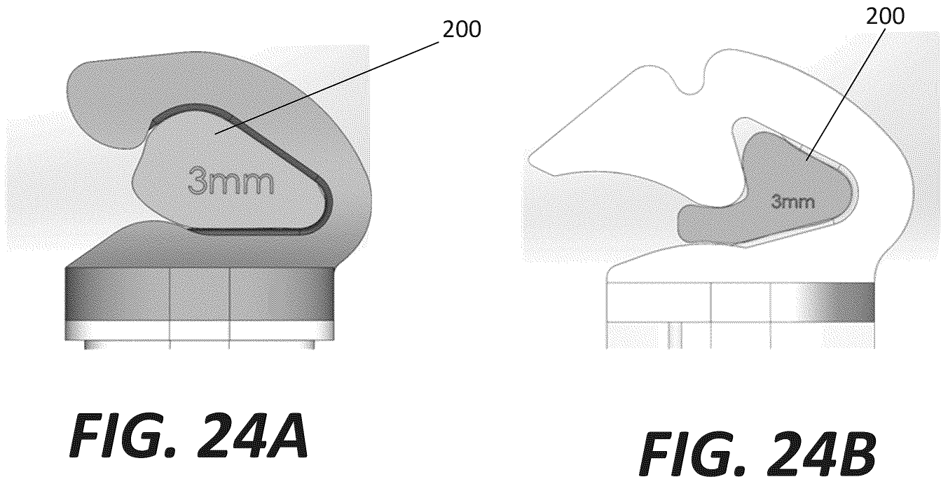

FIGS. 23A-23C schematically show the neuromodulation device in accordance with illustrative embodiments. FIG. 23A shows a 1 mm nerve pressing against the arm 50 and the protrusion 54. In some embodiments, the arm 50 may be movable, and deflects in response to the pressure from the nerve. Additionally, or alternatively, the arm 50 and/or the protrusion 54 is formed of a deformable material (e.g., the buffer layer) that deforms to enlarge the channel gap. In some other embodiments, the arm 50 may be static. FIG. 23B shows the nerve deforming to pass through the continuously axial channel 102. Finally, FIG. 23C shows the nerve 200 positions against electrode 104. Unlike the static arm 50, illustrative embodiments with the movable arm 50 may provide a contact pressure against the nerve 200 that pushes the nerve 200 in contact with the electrode 104.

The nerve shown in FIG. 23C is a relatively small nerve (e.g., 1 mm diameter). Larger nerves (e.g., 3 mm and up) deform to match the available space in the chamber 101 without imparting traumatic pressure along the nerve after reshaping. FIGS. 23D and 23E schematically show large nerves changing their cross-sectional shape but maintaining their cross-sectional area.

Additionally, small nerves 200 are retained in the chamber within very close approximation to the electrodes 104 ensuring the stimulation amplitude can remain low and maintain signal efficiency. Testing by the inventors using the device having one movable arm 50 for large nerves (e.g., 3 mm or greater) found less than 5 mmHg of pressure after seating into the chamber.

It should be apparent that illustrative embodiments provide the advantage of a single device that may couple to and reliably stimulate a variety of different sized nerves. Furthermore, the device may reliably retain nerves of various sizes (e.g., from small 0.5 mm diameter to big 4 mm diameter) without providing overcompression/trauma to the nerves 200. Indeed the chamber 101 (e.g., portion of the movable arm 50) adjusts to assist with retaining the nerve.

Various embodiments use a combination of epoxy and mid-durometer silicone (e.g., Shore A values of 60-90) with or without an additional structural component. For example, the epoxy may be used to support the feedthrough conductors to minimize flexural strain points. The feedthrough conductor (e.g., Pt—Ir, gold, nitinol or similar material) is bent and incorporated into the header structure to provide additional structural support and a holding force. Subsequently, the feedthrough or attached electrode is embedded into a low to mid durometer silicone portion (typically 30-50 Shore A) that has the flexibility to achieve the positioning and resistance needed to hold the nerve in place without causing trauma to the nerve. The structure is unlike typical nerves as the cuff requires both the nerve and the cuff to deform in the extreme cases to “slide” the nerve through the channel and into the chamber. By utilizing the natural ability of the nerve to stretch and the slight motion of the silicone or wire arm of the device, the acute pressure of the nerve is minimized, even in the case of the largest 50-75% larger nerve. Additionally, by utilizing the largest nerves ability to adjust and redistribute it's structure with minimal resting or final force to minimize the internal nerve pressure, there is ample space in the chamber to allow the nerve to relax and achieve a resting pressure well under the 60% compression that is known to cause damage.

For the smallest nerves, the “arm” portion of the device deforms less to achieve the same motion through the channel. In the chamber, there is significantly deformation needed, but the feedthrough bent wire structure as an electrode provides ample opportunity to maintain intimate connection of the nerve and electrode, minimizing impact on signal strength.

Accordingly, in various embodiments, the channel 102 and/or the chamber 101 may be formed of a material configured to deform in response to atraumatic pressures from the nerve (e.g., of less than 6.7 kPa). Additionally, the channel 102 and/or the chamber 101 are configured to change their dimensions as a result of one or more movable arms 50.

Among other advantages, illustrative embodiments the continually axial channel 102 provides easy installation and easy removal of the nerve to and from the chamber 101. The channel 102 may also be non-linear, to assist with retention of the nerve within the channel (so that it does not easily slip out of the continually axial channel). The continually axial channel 102 also assists with reducing trauma to the nerve 200 during the installation and removal procedures. To further assist with reducing trauma, the device may include one or more bending points 81 configured to bend or pivot to accommodate nerves of various shapes and sizes. These features work together to allow the nerve to slide through the channel, and/or to push the arm 50 and/or deform the arm 50 to allow nerve passage.

It should also be apparent that in various embodiments, one or more arms 50 define the size of the chamber 101 and/or the channel 102. The arm 50 additionally provides an atraumatic force that biases nerves 200 of various sizes towards the electrode 104, such that the electrode contacts at least 20%, at least 25%, or at least 35% of the perimeter of the nerve 200. In various embodiments, particularly for larger nerves (e.g., 3 mm or greater), the electrode 104 contacts at least 60% of the nerve perimeter when the nerve 200 is in the chamber 101. The device 100 thus advantageously atraumatically accommodates a variety of different size nerves. Furthermore, the one or more arms 50 aid in continuous contact and electrical coupling with the electrode 104 by providing a restrictive force that presses the nerve. Furthermore, illustrative embodiments couple the device with the nerve without the use of sutures, and without requiring specialized tools, which allows for easy removal of the device.

Although various embodiments describe a device for coupling to, and treating, nerves having diameters of between about 0.5 mm and about 4 mm, it should be understood that such devices and methods are scalable to accommodate a variety of different sized nerves. For example, one skilled in the art may use the disclosure herein to configure the device to couple with larger nerves having diameters of between about 5 mm and about 8 mm. As another example, the device may be configured to couple with nerves 200 having diameters of between about 0.2 mm and about 2 mm. These devices may be configured to couple with the various nerve sizes without damaging the nerve (e.g., by applying pressure to the nerve that is less than 4 kPa).

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Although the subject matter contained herein has been described in detail for the purpose of illustration, it is to be understood that such detail is solely for that purpose and that the present disclosure is not limited to the disclosed embodiments, but, on the contrary, is intended to cover modifications and other arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

Although the subject matter contained herein has been described in detail for the purpose of illustration, it is to be understood that such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

What is claimed is:

1. A neuromodulation device comprising:
a main body comprising a hermetically sealed housing containing electronics therein, and a buffer layer at least partially encapsulating the hermetically sealed housing;
a movable arm coupled with the main body, the movable arm configured to transition between an open configuration and a closed configuration, the movable arm being biased towards the closed configuration;
a nerve stimulation chamber defined at least in part by the movable arm, the nerve stimulation chamber configured to retain a nerve therein, the size and shape of the chamber being adjustable by the movement of the arm in response to contact with the nerve;
an open channel through which the nerve travels, the channel defined at least in part by the movable arm, the size and shape of the channel being adjustable by the movement of the arm in response to contact with the nerve, wherein the channel is continuously axial along a longitudinal axis of the channel when the device the movable arm is in the closed configuration; and
an electrode within the chamber.

2. The neuromodulation device of claim 1, further comprising a second movable arm, the second movable arm configured to transition between an open configuration and a closed configuration, the movable arm being biased towards the closed configuration, wherein movement of the second movable arm adjusts a size of the chamber and/or a size of the channel.

3. The neuromodulation device of claim 1, wherein the movable arm is configured to atraumatically retain a nerve having a resting maximum cross-sectional dimension of between 0.5 mm and 4 mm in the chamber when the movable arm is biased towards the closed configuration.

4. The neuromodulation device of claim 1, wherein the chamber includes one or more nerve relief spaces defined by the movable arm.

5. The neuromodulation device of claim 1, wherein the movable arm includes one or more bending points.

6. The neuromodulation device of claim 1, wherein a distal end of the electrode is embedded within the movable arm.

7. The neuromodulation device of claim 1, wherein a continuous feedthrough electric conductor wire extending through the hermetically sealed housing forms the electrode.

8. The neuromodulation device of claim 7, wherein the channel is non-linear.

9. The neuromodulate device of claim 1, wherein the chamber is configured so that the electrode is curved and sized to contact at least 20% of the perimeter of a nerve having a maximum cross-sectional dimension of between 0.5 mm and 4 mm.

10. The neuromodulation device of claim 1, wherein the channel is non-linear and continuously axial.

11. A method of stimulating a nerve comprising:
providing a neuromodulation device having:
a main body comprising a hermetically sealed housing containing electronics therein, and a buffer layer at least partially encapsulating the hermetically sealed housing,
a movable arm coupled with the main body, the movable arm configured to transition between an open configuration and a closed configuration, the movable arm being biased towards the closed configuration,
a nerve stimulation chamber defined at least in part by the movable arm, the nerve stimulation chamber configured to retain a nerve therein, the size and shape of the chamber being adjustable by the movement of the arm,
a channel through which the nerve travels, the channel defined at least in part by the movable arm, the size and shape of the channel being adjustable by the movement of the arm, wherein the channel is continuously axial along a longitudinal axis of the channel and
an electrode within the chamber;
reducing a maximum cross-sectional dimension of a nerve to define a stretched nerve having a reduced cross-sectional dimension;
moving the stretched nerve through the channel along the central axis;
positioning the nerve in the chamber and increasing the cross-sectional dimension of the stretched nerve.

12. The method as defined by claim 11, further comprising:
transmitting, via the electrode, a control signal to the prescribed nerve
the control signal configured to activate the pelvic floor in a prescribed manner to mitigate the effect of the symptom signal on the spinal cord.

13. The method of claim 11 wherein positioning the prescribed nerve within the chamber contacts the prescribed nerve with the electrode.

14. The method of claim 11 wherein positioning the movable arm in the closed position compresses the prescribed somatic nerve in the chamber, such that the cross-sectional shape of the nerve changes without damaging the nerve.

15. The method of claim 11 wherein the housing includes a projection configured to press against the movable arm in the closed position, the projection configured to limit the amount of compression on the nerve from the movable arm.

16. The method of claim 11 wherein a continuous feedthrough conductor extending through the hermetically sealed housing forms the electrode.

17. The method of claim 11 wherein the electrode contacts more than 50 percent of a perimeter of the nerve.

18. The method of claim 11 wherein the movable arm is biased towards the closed position.

19. The method of claim 11 wherein the prescribed somatic motor nerve comprises the perineal nerve, further wherein the perineal or pelvic floor nerve is part of the coccygeal and/or perineal plexus.

20. The method of claim 11 wherein transmitting comprises using a signal generator to produce and transmit the symptom control signal with the electrode, the symptom control signal being transmitted using one or more of wired or wireless communication media.
